(19) United States
(12) Patent Application Publication
(10) Pub. No.: US 2012/0052066 A1
(43) Pub. Date: Mar. 1, 2012

(54) MARKERS AND METHODS FOR ASSESSING AND TREATING LUPUS PATIENTS SUSCEPTIBLE TO PHOTOPROVOCATION

(76) Inventors: Cesar Calderon, Radnor, PA (US); John Getsy, Plymouth Meeting, PA (US)

(21) Appl. No.: 13/127,221

(22) PCT Filed: Nov. 6, 2009
(86) PCT No.: PCT/US09/63554

§ 371 Date: Nov. 2, 2011

Related U.S. Application Data
(60) Provisional application No. 61/112,386, filed on Nov. 7, 2008.

(51) Int. Cl. A61K 39/395; C12Q 1/68; A61P 37/02; C40B 40/06; C40B 40/10; C40B 30/00; G01N 33/566

(52) U.S. Cl. 424/134.1; 506/7; 435/6.12; 435/7.92; 506/16; 506/18

(57) ABSTRACT

A method for predicting or detecting susceptibility to lupus of an individual subjected to photoprovocation obtains biological samples from the individual before and after exposure to photoprovocation and compares the levels of at least a portion of members of a 45-member panel or subset thereof to determine whether the individual is susceptible to lupus. The method enables identification of potential lupus patients prior to onset of disease symptoms.

### CROSS-REFERENCE TO RELATED APPLICATIONS

This application claims the benefit of International Application Number PCT/US2009/063554, filed 6 Nov. 2009, which claims the benefit of U.S. Provisional Application No. 61/112,386, filed 7 Nov. 2008. The entire contents of each of the aforesaid applications are incorporated herein by reference in their entirety.

### FIELD OF THE INVENTION

The invention relates to the identification of expression profiles and the nucleic acids indicative of susceptibility to and protection from lupus, and to the use of such expression profiles and nucleic acids in diagnosis and treatment of lupus. The invention further relates to methods for identifying, using, and testing susceptibility to lupus due to photoprovocation or other causes.

### BACKGROUND OF THE INVENTION

Cutaneous Lupus Erythematosus (CLE) is the focus of intense research as scientists try to determine what causes the disease and how to best be treat it. Some of the questions that need to be answered include who gets lupus, and why, and why are women more likely than men to have the disease? Why are there more cases of lupus in some racial and ethnic groups? What goes wrong in the immune system and why? How can we correct the way the immune system functions once something goes wrong? What treatment approaches will work best to lessen or cure lupus symptoms?

To help answer these questions, scientists are developing new and better ways to study the disease. They are performing laboratory studies that compare various aspects of the immune system of people with lupus with those without lupus. They also use mice with disorders resembling lupus to better understand the abnormalities of the immune system that occur in lupus and to identify possible new therapies. The main problems in the actual approach for the study of lupus are:
1) The unpredictability of disease onset and progression makes the collection of meaningful biomarkers in longitudinal studies very difficult.
2) The use of animal models with “lupus like” syndrome becomes even less relevant as a research tool when the information obtained from such models does not translate into humans.
3) Considerable resources are devoted to the identification of the following type of biomarkers:
   Proteins: Applicable throughout the drug discovery and development process; longest history as biomarkers
   RNA Expression Profiling: Gene arrays used in discovery phase and clinical trials
   SNPs (Single Nucleotide Polymorphisms): Large patient population requirements have hindered use, but should increase in the future.

Cell-based Proteins: In particular cell surface markers. Large patient populations required

Nevertheless, no useful biomarker for drug development in lupus has been developed using the technologies listed above.

Photoprovocation permits the development of skin lesions in a controlled and safe manner by exposing the subject to a personalized amount of triggering UVA/UVB radiation. These effects of photoprovocation can be studied to assess CLE parameters. Taking into consideration that lesion development in CLE is seasonal and depends on both the intensity of ultraviolet radiation and the degree of the individual's sensitivity to light, photoprovocation can be used to trigger, in a controlled way, the disease in cohort of subjects at the same time.

Considering the high enzymatic activity in the dynamics of lupus, Peptidomics yields stable molecules containing information about the disease process.

Microarray technology is a powerful tool since it enables analysis of the expression of thousands of genes simultaneously and can also be automated allowing for a high-throughput format. In diseases associated with complex host functions, such as those known as immune mediated inflammatory diseases, such as lupus, microarray results can provide a gene expression profile that can be of utility in designing new approaches to disease diagnosis and management. These approaches also serve to identify novel genes and annotating genes of unknown function heretofore unassociated with the disease or condition. Accordingly, there is a need to identify and characterize new gene markers useful in developing methods for identifying a subject's risk of developing inflammatory disorders, such as lupus, as well as other diseases and conditions.

Gene expression can be modulated in several different ways, including by the use of siRNAs, shRNAs, antisense molecules and DNAzymes. SiRNAs and shRNAs both work via the RNAi pathway and have been successfully used to suppress the expression of genes. RNAi was first discovered in worms and the phenomenon of gene silencing related to dsRNA was first reported in plants by Fire and Mello and is thought to be a way for plant cells to combat infection with RNA viruses. In this pathway, the long dsRNA viral product is processed into smaller fragments of 21-25 by in length by a DICER-like enzyme and then the double-stranded molecule is unwound and loaded into the RNA induced silencing complex (RISC). A similar pathway has been identified in mammalian cells with the notable difference that the dsRNA molecules must be smaller than 30 by in length in order to avoid the induction of the so-called interferon response, which is not gene specific and leads to the global shut down of protein synthesis in the cell.

Synthetic siRNAs have been successfully designed to selectively target a single gene and can be delivered to cells in vitro or in vivo. ShRNAs are the DNA equivalents of siRNA molecules and have the advantage of being incorporated into a cells' genome where they are replicated during every mitotic cycle.

DNAzymes have also been used to modulate gene expression. DNAzymes are catalytic DNA molecules that cleave single-stranded RNA. They are highly selective for the target RNA sequence and as such can be used to down-regulate specific genes through targeting of the messenger RNA.

Accordingly, there is a need to identify and characterize new gene markers useful in developing methods for identifying the susceptibility of a subject to a inflammatory disorder, such as lupus, caused by, for example, photoprovocation.

### SUMMARY OF THE INVENTION

The present invention relates to a method for detecting susceptibility to lupus, such as CLE, in a subject. The invention further comprises a method for predicting whether a lupus (CLE) patient will respond to treatment with a therapeutic agent. The methods of the invention comprise measuring levels of expression of peptides in subject samples before and after exposure to photoprovocation and, optionally, before and after treatment with a therapeutic agent, and correlating the relative levels to whether a subject is susceptible to lupus and its symptoms. The modified expression levels constitute a profile that can serve as a biomarker profile predictive of a patient's susceptibility to lupus and/or responsiveness to potential treatment. In one embodiment, the sample is from the skin of a subject (e.g., biopsy); in another embodiment, the sample is from the plasma of a subject.

In one embodiment of the invention, the profile is used as a human disease model and a biomarker tool that are complementary to each other: the application of photoprovocation in subjects diagnosed with CLE and a unique peptidomics platform that uses mass spectrometry and projection to latent structures for multivariate data analysis for the identification of biomarkers. The present invention allows correlating data points from baseline to last patient visit to make predictions for subjects. Taking into consideration that lesion development in CLE is seasonal and depends on both the intensity of ultraviolet radiation and the degree of the individual's sensitivity to light, the method of the invention can be used to trigger, in a controlled way the disease in a cohort of subjects at the same time.

Optionally, statistical analysis is performed on the changes in levels of members of the gene panel to evaluate the significance of these changes and to identify which members are meaningful members of the panel.

In an alternative embodiment, the present invention comprises a kit for predicting the susceptibility to lupus or its symptoms based on the pattern of peptide expression.

The present invention further provides any invention described herein.

### BRIEF DESCRIPTION OF THE DRAWINGS

FIG. 1 is the skin peptidome in response to the experimental design (PLS components 3 and 4) Biplot of the weights for the 13,835 signal coordinates (grey points), and of the Y-loadings for the relevant experimental variables. Black dots indicate signals, which were significantly regulated when compared to the zero distribution.

FIG. 2 is the skin peptidome in response to the experimental design (PLS components 5 and 6) Biplot of the weights for the 13,835 signal coordinates (grey points), and of the Y-loadings for the relevant experimental variables. Black dots indicate signals, which were significantly regulated when compared to the zero distribution.

FIG. 3 is the plasma peptidome in response to the experimental design (PLS components 3 and 4) Biplot of the weights for the 25,383 signal coordinates (grey points), and of the Y-loadings for the relevant experimental variables (vectors). Black dots indicate signals, which were significantly regulated when compared to the zero distribution

FIG. 4 is the plasma peptidome in response to the experimental design (PLS components 5 and 6) Biplot of the weights for the 25,383 signal coordinates (grey points), and of the Y-loadings for the relevant experimental variables (vectors). Black dots indicate signals, which were significantly regulated when compared to the zero distribution

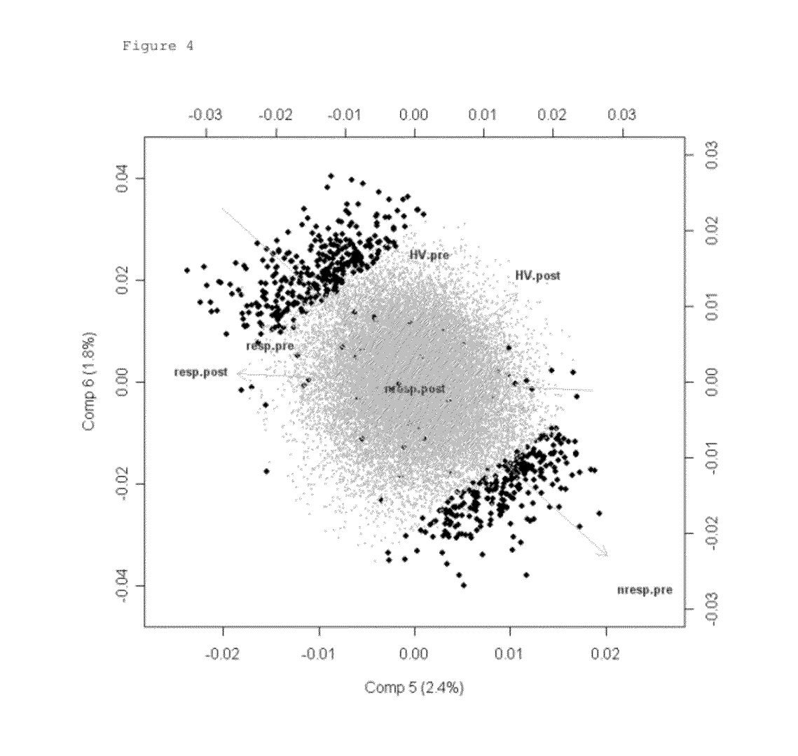

FIG. 5 is the identification of a peptide from the CD99 antigen precursor molecule. The peptide was identified using MS/MS analysis and the MASCOT search engine identifying the sequence tag [VT]GAVVVA.

### DETAILED DESCRIPTION OF THE INVENTION

### Definitions

The following definitions are set forth to illustrate and define the meaning and scope of various terms used to describe the invention herein.

An “activity,” a biological activity, and a functional activity of a polypeptide refers to an activity exerted by a gene of the gene panel in response to its specific interaction with another protein or molecule as determined in vivo, in situ, or in vitro, according to standard techniques. Such activities can be a direct activity, such as an association with or an enzymatic activity on a second protein, or an indirect activity, such as a cellular process mediated by interaction of the protein with a second protein or a series of interactions as in intracellular signaling or the coagulation cascade.

An “antibody” includes any polypeptide or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion, fragment or variant thereof. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. For example, antibody fragments include, but are not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, and single domain antibodies (e.g., V_{H} or V_{L}), are encompassed by the invention (see, e.g., Colligan, et al., eds., Current Protocols in Immunology, John Wiley & Sons, Inc., NY (1994-2001); Colligan et al., Current Protocols in Polypeptide Science, John Wiley & Sons, NY (1997-2001)).

The terms “array” or “microarray” or “biochip” or “chip” as used herein refer to articles of manufacture or devices comprising a plurality of immobilized target elements, each target element comprising a “clone,” “feature,” “spot” or defined area comprising a particular composition, such as a biological molecule, e.g., a nucleic acid molecule or polypeptide, immobilized to a solid surface, as discussed in further detail, below.

“Complement of” or “complementary to” a nucleic acid sequence of the invention refers to a polynucleotide molecule having a complementary base sequence and reverse orientation as compared to a first polynucleotide.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing:Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.).

The terms “specifically hybridize to,” “hybridizing specifically to,” “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence; and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Alternative hybridization conditions that can be used to practice the invention are described in detail, below. In alternative aspects, the hybridization and/or wash conditions are carried out under moderate conditions, stringent conditions and very stringent conditions, as described in further detail, below. Alternative wash conditions are also used in different aspects, as described in further detail, herein.

The phrases “labeled biological molecule” or “labeled with a detectable composition” or “labeled with a detectable moiety” as used herein refer to a biological molecule, e.g., a nucleic acid, comprising a detectable composition, i.e., a label, as described in detail, below.

The label can also be another biological molecule, as a nucleic acid, e.g., a nucleic acid in the form of a stem-loop structure as a “molecular beacon,” as described below. This includes incorporation of labeled bases (or, bases which can bind to a detectable label) into the nucleic acid by, e.g., nick translation, random primer extension, amplification with degenerate primers, and the like. Any label can be used, e.g., chemiluminescent labels, radiolabels, enzymatic labels and the like. The label can be detectable by any means, e.g., visual, spectroscopic, photochemical, biochemical, immunochemical, physical, chemical and/or chemiluminescent detection. The invention can use arrays comprising immobilized nucleic acids comprising detectable labels.

The term “nucleic acid” as used herein refers to a deoxyribonucleotide (DNA) or ribonucleotide (RNA) in either single- or double-stranded form. The term encompasses nucleic acids containing known analogues of natural nucleotides. The term nucleic acid is used interchangeably with gene, DNA, RNA, cDNA, mRNA, oligonucleotide primer, probe and amplification product. The term also encompasses DNA backbone analogues, such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs).

The terms “sample” as used herein refer to a sample comprising a DNA or RNA, or nucleic acid representative of DNA or RNA or peptide isolated from a natural source. A “sample of nucleic acids” is in a form suitable for hybridization (e.g., as a soluble aqueous solution) to another nucleic acid (e.g., immobilized probes). The sample nucleic acid may be isolated, cloned, or extracted from particular cells or tissues. The cell or tissue sample from which the nucleic acid sample is prepared is typically taken from a patient having or suspected of having CLE or a related disease or condition. Methods of isolating cell and tissue samples are well known to those of skill in the art and include, but are not limited to, punch biopsies, aspirations, tissue sections, needle biopsies, and the like. Frequently the sample will be a “clinical sample” which is a sample derived from a patient, including sections of tissues such as frozen sections or paraffin sections taken for histological purposes. The sample can also be derived from plasma, supernatants (of cells) or the cells themselves taken from patients or from cell cultures, cells from tissue culture and other media in which it may be desirable to detect the response to drug candidates. In some cases, the nucleic acids may be amplified using standard techniques such as PCR, prior to the hybridization. The probe can be produced from and collectively can be representative of a source of nucleic acids from one or more particular (pre-selected) portions of, e.g., a collection of polymerase chain reaction (PCR) amplification products, substantially an entire chromosome or a chromosome fragment, or substantially an entire genome, e.g., as a collection of clones, e.g., BACs, PACs, YACs, and the like (see below).

“Nucleic acids” are polymers of nucleotides, wherein a nucleotide comprises a base linked to a sugar which sugars are in turn linked one to another by an interceding at least bivalent molecule, such as phosphoric acid. In naturally occurring nucleic acids, the sugar is either 2′-deoxyribose (DNA) or ribose (RNA). Unnatural poly- or oliogonucleotides contain modified bases, sugars, or linking molecules, but are generally understood to mimic the complementary nature of the naturally occurring nucleic acids after which they are designed. An example of an unnatural oligonucleotide is an antisense molecule composition that has a phosphorothiorate backbone. An “oligonucleotide” generally refers to a nucleic acid molecule having less than 30 nucleotides.

The term “profile” means a pattern and relates to the magnitude and direction of change of a number of features. The profile may be interpreted stringently, i.e., where the variation in the magnitude and/or number of features within the profile displaying the characteristic is substantially similar to a reference profile or it may be interpreted less stringently, for example, by requiring a trend rather than an absolute match of all or a subset of feature characteristics.

The terms “protein,” “polypeptide,” and “peptide” include “analogs,” or “conservative variants” and “mimetics” or “peptidomimetics” with structures and activity that substantially correspond to the polypeptide from which the variant was derived, as discussed in detail above.

A “polypeptide” or “peptide” is a polymer of amino acid residues joined by peptide bonds, and a peptide generally refers to amino acid polymers of 12 or less residues. Peptide bonds can be produced naturally as directed by the nucleic acid template or synthetically by methods well known in the art.

A “protein” is a macromolecule comprising one or more polypeptide chains. A protein may further comprise substituent groups attached to the side groups of the amino acids not involved in formation of the peptide bonds. Typically, proteins formed by eukaryotic cell expression also contain carbohydrates. Proteins are defined herein in terms of their amino acid sequence or backbone and substituents are not specified, whether known or not.

The term “receptor” denotes a molecule having the ability to affect biological activity, in e.g., a cell, as a result of interaction with a specific ligand or binding partner. Cell membrane bound receptors are characterized by an extracellular ligand-binding domain, one or more membrane spanning or transmembrane domains, and an intracellular effector domain that is typically involved in signal transduction. Ligand binding to cell membrane receptors causes changes in the extracellular domain that are communicated across the cell membrane, direct or indirect interaction with one or more intracellular proteins, and alters cellular properties, such as enzyme activity, cell shape, or gene expression profile. Receptors may also be untethered to the cell surface and may be cytosolic, nuclear, or released from the cell altogether. Non-cell associated receptors are termed soluble receptors or ligands.

All publications or patents cited herein are entirely incorporated herein by reference, whether or not specifically designated accordingly, as they show the state of the art at the time of the present invention and/or provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY (1987-2001); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY (1997-2001).

### Gene Panel Identification and Validation

The present invention provides novel methods for detecting or predicting susceptibility to lupus (e.g., CLE) and its symptoms, e.g., skin lesions, and detecting or predicting a lupus subject's response to a potential therapy.

In one aspect, the expression levels of genes or peptides are determined in different patient samples for which diagnosis information is desired, to provide profiles. A profile of a particular sample is essentially a “fingerprint” of the state of the sample; while two states may have any particular peptide similarly expressed, the evaluation of a number of peptides simultaneously allows the generation of a profile that is unique to the state of the patient sample. That is, normal tissue may be distinguished from lesion tissue and tissue from a treated patient may be distinguished from an untreated patient. By comparing profiles of tissue in different disease states that are known, information regarding which peptides are important (including both up- and down-regulation of peptides/genes) in each of these states is obtained.

The identification of sequences (peptides) that are differentially expressed in disease tissue allows the use of this information in a number of ways. For example, the evaluation of a particular treatment regime may be evaluated.

This may be done by making biochips comprising sets of the important disease genes, which can then be used in these screens. These methods can also be performed on the protein basis; that is, protein expression levels of the lupus-related gene product proteins can be evaluated for diagnostic purposes or to screen candidate agents. In addition, the nucleic acid or peptide sequences comprising the lupus-related gene profile can be used to measure whether a patient is likely to respond to a therapeutic prior to treatment.

Lupus-related gene sequences can include both nucleic acid and amino acid sequences. In one embodiment, the lupus-related profile are peptides. In another embodiment, the lupus-related gene sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus, an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention.

### Method of Practicing the Invention

The invention provides in silico, array-based methods relying on the relative amount of a binding molecule (e.g., nucleic acid sequence) in two or more samples. Also provided are computer-implemented methods for determining the relative amount of a binding molecule (e.g., nucleic acid sequence) in two or more samples and using the determined relative binding amount to predict responsiveness to a particular therapy, and monitor and enhance therapeutic treatment.

In an embodiment of the methods of the invention, two or more samples of labeled biological molecules are applied to two or more arrays, where the arrays have substantially the same complement of immobilized binding molecule (e.g., immobilized nucleic acid capable of hybridizing to labeled sample nucleic acid). The two or more arrays are typically multiple copies of the same array. However, because each “spot,” “clone” or “feature” on the array has similar biological molecules (e.g., nucleic acids of the same sequence) and the biological molecules (e.g., nucleic acid) in each spot is known, as is typical of nucleic acid and other arrays, it is not necessary that the multiple arrays used in the invention be identical in configuration it is only necessary that the position of each feature on the substrate be known, that is, have an address. Thus, in one aspect, multiple biological molecules (e.g., nucleic acid) in samples are comparatively bound to the array (e.g., hybridized simultaneously) and the information gathered is coded so that the results are based on the inherent properties of the feature (e.g., the nucleic acid sequence) and not it's position on the substrate.

Amplification of Nucleic Acids

Amplification using oligonucleotide primers can be used to generate nucleic acids used in the compositions and methods of the invention, to detect or measure levels of test or control samples hybridized to an array, and the like. The skilled artisan can select and design suitable oligonucleotide amplification primers. Amplification methods are also well known in the art, and include, e.g., polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Hybridizing Nucleic Acids

In practicing the methods of the invention, test and control samples of nucleic acid are hybridized to immobilized probe nucleic acid, e.g., on arrays. In alternative aspects, the hybridization and/or wash conditions are carried out under moderate conditions, stringent conditions and very stringent conditions. An extensive guide to the hybridization of nucleic acids is found in, e.g., Sambrook Ausubel, Tijssen. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or a filter in a Southern or northern blot is 42° C. using standard hybridization solutions (see, e.g., Sambrook), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook). Often, a high stringency wash is preceded by a medium or low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

In alternative aspects of the compositions and methods of the invention, e.g., in practicing comparative nucleic acid hybridization, such as comparative genomic hybridization (CGH) with arrays, the fluorescent dyes Cy3® and Cy5® are used to differentially label nucleic acid fragments from two samples, e.g., the array-immobilized nucleic acid versus the sample nucleic acid, or, nucleic acid generated from a control versus a test cell or tissue. Many commercial instruments are designed to accommodate the detection of these two dyes. To increase the stability of Cy50, or fluors or other oxidation-sensitive compounds, antioxidants and free radical scavengers can be used in hybridization mixes, the hybridization and/or the wash solutions. Thus, Cy50 signals are dramatically increased and longer hybridization times are possible. See WO 0194630 A2 and U.S. Patent Application No. 20020006622.

To further increase the hybridization sensitivity, hybridization can be carried out in a controlled, unsaturated humidity environment; thus, hybridization efficiency is significantly improved if the humidity is not saturated. See WO 0194630 A2 and U.S. Patent Application No. 20020006622. The hybridization efficiency can be improved if the humidity is dynamically controlled, i.e., if the humidity changes during hybridization. Mass transfer will be facilitated in a dynamically balanced humidity environment. The humidity in the hybridization environment can be adjusted stepwise or continuously. Array devices comprising housings and controls that allow the operator to control the humidity during pre-hybridization, hybridization, wash and/or detection stages can be used. The device can have detection, control and memory components to allow pre-programming of the humidity and temperature controls (which are constant and precise or which flucturate), and other parameters during the entire procedural cycle, including pre-hybridization, hybridization, wash and detection steps. See WO 0194630 A2 and U.S. Patent Application No. 20020006622.

The methods of the invention can comprise hybridization conditions comprising osmotic fluctuation. Hybridization efficiency (i.e., time to equilibrium) can also be enhanced by a hybridization environment that comprises changing hyper-/hypo-tonicity, e.g., a solute gradient. A solute gradient is created in the device. For example, a low salt hybridization solution is placed on one side of the array hybridization chamber and a higher salt buffer is placed on the other side to generate a solute gradient in the chamber. See WO 0194630 A2 and U.S. Patent Application No. 20020006622.

Blocking the Ability of Repetitive Nucleic Acid Sequences to Hybridize

The methods of the invention can comprise a step of blocking the ability of repetitive nucleic acid sequences to hybridize (i.e., blocking “hybridization capacity”) in the immobilized nucleic acid segments. The hybridization capacity of repetitive nucleic acid sequences in the sample nucleic acid sequences can be blocked by mixing sample nucleic acid sequences with unlabeled or alternatively labeled repetitive nucleic acid sequences. Sample nucleic acid sequences can be mixed with repetitive nucleic acid sequences before the step of contacting with the array-immobilized nucleic acid segments. Blocking sequences are for example, Cot-1 DNA, salmon sperm DNA, or specifc repetitive genomic sequences. The repetitive nucleic acid sequences can be unlabeled. A number of methods for removing and/or disabling the hybridization capacity of repetitive sequences using, e.g., Cot-1 are known; see, e.g., Craig (1997) Hum. Genet. 100:472-476; WO 93/18186. Repetitive DNA sequences can be removed from library probes by means of magnetic purification and affinity PCR, see, e.g., Rauch (2000) J. Biochem. Biophys. Methods 44:59-72.

Arrays are generically a plurality of target elements immobilized onto the surface of the plate as defined “spots” or “clusters,” or “features,” with each target element comprising one or more biological molecules (e.g., nucleic acids or polypeptides) immobilized to a solid surface for specific binding (e.g., hybridization) to a molecule in a sample. The immobilized nucleic acids can contain sequences from specific messages (e.g., as cDNA libraries) or genes (e.g., genomic libraries), including a human genome. Other target elements can contain reference sequences and the like. The biological molecules of the arrays may be arranged on the solid surface at different sizes and different densities. The densities of the biological molecules in a cluster and the number of clusters on the array will depend upon a number of factors, such as the nature of the label, the solid support, the degree of hydrophobicity of the substrate surface, and the like. Each feature may comprise substantially the same biological molecule (e.g., nucleic acid), or, a mixture of biological molecules (e.g., nucleic acids of different lengths and/or sequences). Thus, for example, a feature may contain more than one copy of a cloned piece of DNA, and each copy may be broken into fragments of different lengths.

Array substrate surfaces onto which biological molecules (e.g., nucleic acids) are immobilized can include nitrocellulose, glass, quartz, fused silica, plastics and the like, as discussed further, below. The compositions and methods of the invention can incorporate in whole or in part designs of arrays, and associated components and methods, as described, e.g., in U.S. Pat. Nos. 6,344,316; 6,197,503; 6,174,684; 6,159,685; 6,156,501; 6,093,370; 6,087,112; 6,087,103; 6,087,102; 6,083,697; 6,080,585; 6,054,270; 6,048,695; 6,045,996; 6,022,963; 6,013,440; 5,959,098; 5,856,174; 5,843,655; 5,837,832; 5,770,456; 5,723,320; 5,700,637; 5,695, 940; 5,556,752; 5,143,854; see also, e.g., WO 99/51773; WO 99/09217; WO 97/46313; WO 96/17958; WO 89/10977; see also, e.g., Johnston (1998) Curr. Biol. 8:R171-174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32; Epstein (2000) Current Opinion in Biotech. 11:36-41; Mendoza (1999 Biotechniques 27: 778-788; Lueking (1999) Anal. Biochem. 270:103-111; Davies (1999) Biotechniques 27:1258-1261.

Substrate Surfaces

Substrate surfaces that can be used in the compositions and methods of the invention include, for example, glass (see, e.g., U.S. Pat. No. 5,843,767), ceramics, and quartz. The arrays can have substrate surfaces of a rigid, semi-rigid or flexible material. The substrate surface can be flat or planar, be shaped as wells, raised regions, etched trenches, pores, beads, filaments, or the like. Substrate surfaces can also comprise various materials such as nitrocellulose, paper, crystalline substrates (e.g., gallium arsenide), metals, metalloids, polacryloylmorpholide, various plastics and plastic copolymers, Nylon®, Teflon®, polyethylene, polypropylene, latex, polymethacrylate, poly (ethylene terephthalate), rayon, nylon, poly(vinyl butyrate), and cellulose acetate. The substrates may be coated and the substrate and the coating may be functionalized to, e.g., enable conjugation to an amine.

Arrays Comprising Calibration Sequences

The invention comtemplates the use of arrays comprising immobilized calibration sequences for normalizing the results of array-based hybridization reactions, and methods for using these calibration sequences, e.g., to determine the copy number of a calibration sequence to “normalize” or “calibrate” ratio profiles. The calibration sequences can be substantially the same as a unique sequence in an immobilized nucleic acid sequence on an array. For example, a “marker” sequence from each “spot” or “biosite” on an array (which is present only on that spot, making it a “marker” for that spot) is represented by a corresponding sequence on one or more “control” or “calibration” spot(s).

The “control spots” or “calibration spots” are used for “normalization” to provide information that is reliable and repeatable. Control spots can provide a consistent result independent of the labeled sample hybridized to the array (or a labeled binding molecule from a sample). The control spots can be used to generate a “normalization” or “calibration” curve to offset possible intensity errors between the two arrays (or more) used in the in silico, array-based methods of the invention.

One method of generating a control on the array would be to use an equimolar mixture of all the biological molecules (e.g., nucleic acid sequences) spotted on the array and generating a single spot. This single spot would have equal amounts of the biological molecules (e.g., nucleic acid sequences) from all the other spots on the array. Multiple control spots can be generated by varying the concentration of the equimolar mixture.

### Samples and Specimens

The sample peptides may be isolated, cloned, or extracted from particular cells, tissues, or other specimens. The cell or tissue sample from which the peptide sample is prepared is typically taken from a patient having or suspected of having lupus or a related condition. Methods of isolating cell and tissue samples are well known to those of skill in the art and include, but are not limited to, aspirations, punch biopsies, tissue sections, needle biopsies, and the like. Frequently, the sample will be a “clinical sample” which is a sample derived from a patient, including whole blood, or sections of tissues, such as frozen sections or paraffin sections taken for histological purposes. The sample can also be derived from supernatants (of cells) or the cells themselves taken from patients or from cell cultures, cells from tissue culture and other media in which it may be desirable to detect the response to drug candidates. In some cases, the nucleic acids may be amplified using standard techniques such as PCR, prior to the hybridization.

In one embodiment, the present invention is a pre-treatment method of predicting disease regression or resolution. The method includes (1) taking a skin biopsy or other specimen from an individual diagnosed with lupus or a related disease or disorder, (2) measuring the levels of the profile peptides of the panel, (3) comparing the pre-treatment level of the peptides with a pre-treatment reference profile from treatment responders, and (4) predicting treatment response by monitoring the levels of the peptide panel.

### Methods of Assessing Biomarker Utility

The prognostic utility of the present biomarker gene panel for assessing a patient's response to treatment or prognosis of disease can be validated by using other means for assessing a patient's state of disease. For example, gross measurement of disease may be assessed and recorded by certain imaging methods, such as but not limited to: imaging by photographic, radiometric, or magnetic resonance technology. General indices of health or disease further include serum or blood composition (protein, liver enzymes, pH, electrolytes, red cell volume, hematocrit, hemoglobin, or specific protein).

### Patient Assessment and Monitoring

Some of the peptides in the panel belong to classes of peptides that have been reported to be aberrantly expressed in lupus patients previously, such as ???, the expression patterns of the genes over the course of treatment have not been studied in the treatment of lupus, and none has been identified as having predictive value. The panel of biomarkers disclosed herein permits the generation of methods for rapid and reliable prediction, diagnostic tools that predict the clinical outcome of a lupus trial, or prognostic tools for tracking the efficacy of lups therapy. Prognostic methods based on detecting these peptides in a sample are provided. These compositions may be used, for example, in connection with the diagnosis, prevention and treatment of a range of immune-mediated inflammatory diseases. For example, kininogen and CD99 peptides may block migration and/or diapedesis of lymphocytes and/or leukocytes in inflammatory processes such that their detection as biomarkers is relevant in related disorders.

### Therapeutic Agents

### Antagonists

As used herein, the term “antagonists” refer to substances which inhibit or neutralize the biologic activity of the product of the lupus-related panel of the invention. Such antagonists accomplish this effect in a variety of ways. One class of antagonists will bind to the peptide or protein with sufficient affinity and specificity to neutralize the biologic effects of the protein. Included in this class of molecules are antibodies and antibody fragments (such as, for example, F(ab) or F(ab′)₂ molecules). Another class of antagonists comprises fragments of the protein, muteins or small organic molecules, i.e., peptidomimetics, that will bind to the cognate binding partners or ligands of the protein, thereby inhibiting the biologic activity of the specific interaction of the protein with its cognate ligand or receptor. The lupus-related antagonist may be of any of these classes as long as it is a substance that inhibits at least one biological activity of the protein.

Antagonists include antibodies directed to one or more regions of the protein or fragments thereof, antibodies directed to the cognate ligand or receptor, and partial peptides of the protein or its cognate ligand which inhibit at least one biological activity of the protein. Another class of antagonists includes siRNAs, shRNAs, antisense molecules and DNAzymes targeting the gene sequence as known in the art are disclosed herein.

Suitable antibodies include those that compete for binding to lupus-related proteins with monoclonal antibodies that block lupus-related protein activation or prevent the lupus-related protein from binding to its cognate ligand, or prevent lupus-related protein signalling.

A therapeutic targeting the inducer of the lupus-related protein may provide better chances of success. Gene expression can be modulated in several different ways including by the use of siRNAs, shRNAs, antisense molecules and DNAzymes. Synthetic siRNAs, shRNAs, and DNAzymes can be designed to specifically target one or more genes and they can easily be delivered to cells in vitro or in vivo.

The present invention encompasses antisense nucleic acid molecules, i.e., molecules that are complementary to a sense nucleic acid encoding a lupus-related polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a UC-related gene product polypeptide. The non-coding regions (“5′ and 3′ untranslated regions”) are the 5′ and 3′ sequences that flank the coding region and are not translated into amino acids.

The invention also provides chimeric or fusion proteins. As used herein, a “chimeric protein” or “fusion protein” comprises all or part (preferably biologically active) of a lupus-related polypeptide operably linked to a heterologous polypeptide (i.e., a polypeptide other than the same lupus-related polypeptide). Within the fusion protein, the term “operably linked” is intended to indicate that the lupus-related polypeptide and the heterologous polypeptide are fused in-frame to each other. The heterologous polypeptide can be fused to the amino-terminus or the carboxyl-terminus of the lupus-related polypeptide. In another embodiment, a lupus-related polypeptide or a domain or active fragment thereof can be fused with a heterologous protein sequence or fragment thereof to form a chimeric protein, where the polypeptides, domains or fragments are not fused end to end but are interposed within the heterologous protein framework.

In yet another embodiment, the fusion protein is an immunoglobulin fusion protein in which all or part of a lupus-related polypeptide is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a lupus-related polypeptide. Inhibition of ligand/receptor interaction can be useful therapeutically, both for treating proliferative and differentiative disorders and for modulating (e.g., promoting or inhibiting) cell survival. A preferred embodiment of an immunoglobulin chimeric protein is a C_{H}1 domain-deleted immunoglobulin or MIMETIBODY™ construct having an active polypeptide fragment interposed within a modified framework region as taught in co-pending application PCT WO/04002417. Moreover, the immunoglobulin fusion proteins of the invention can be used as immunogens to produce antibodies directed against a lupus-related polypeptide in a subject, to purify ligands and in screening assays to identify molecules that inhibit the interaction of receptors with ligands.

### Compositions and Their Uses

In accordance with the invention, the neutralizing anti-lupus-related protein antagonists, such as monoclonal antibodies, described herein can be used to inhibit lupus-related protein activity. Additionally, such antagonists can be used to inhibit the pathogenesis of lupus and -related inflammatory diseases amenable to such treatment. The individual to be treated may be any mammal and is preferably a primate, a companion animal which is a mammal and, most preferably, a human patient. The amount of antagonist administered will vary according to the purpose it is being used for and the method of administration.

The lupus-related protein antagonists may be administered by any number of methods that result in an effect in tissue in which pathological activity is desired to be prevented or halted. Further, the anti-lupus-related protein antagonists need not be present locally to impart an effect on the lupus-related protein activity, therefore, they may be administered wherever access to body compartments or fluids containing lupus-related protein is achieved. In the case of inflamed, malignant, or otherwise compromised tissues, these methods may include direct application of a formulation containing the antagonists. Such methods include intravenous administration of a liquid composition, transdermal administration of a liquid or solid formulation, oral, topical administration, or interstitial or inter-operative administration. Adminstration may be affected by the implantation of a device whose primary function may not be as a drug delivery vehicle.

For antibodies, the preferred dosage is about 0.1 mg/kg to 100 mg/kg of body weight (generally about 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, the use of lower dosages and less frequent administration is often possible. Modifications, such as lipidation, can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The lupus-related protein antagonist nucleic acid molecules can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054- 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Pharmacogenomics

Agents, or modulators that have a stimulatory or inhibitory effect on activity or expression of a lupus-related polypeptide as identified by a screening assay described herein, can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a lupus-related polypeptide, expression of a lupus-related protein encoding nucleic acid, or mutation content of a lupus-related protein gene in an individual can be determined to thereby select an appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2): 254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so- called ultra-rapid metabolizers who do not respond to standard doses. Recently, the molecular basis of ultra-rapid metabolism has been identified to be due to CYP2D6 gene amplification.

Thus, the activity of a lupus-related polypeptide, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

### Methods of Treatment

The present invention provides for both prophylactic and therapeutic methods of treating a subject at risk of (or susceptible to) a disorder or having a disorder associated with aberrant expression or activity of a lupus-related polypeptide and/or in which the lupus-related polypeptide is involved.

The present invention provides a method for modulating or treating at least one lupus-related protein related disease or condition, in a cell, tissue, organ, animal, or patient, as known in the art or as described herein, using at least one lupus-related protein antagonist.

Compositions of lupus-related protein antagonist may find therapeutic use in the treatment of lupus or related conditions. The present invention also provides a method for modulating or treating at least one lupus-related disease, in a cell, tissue, organ, animal, or patient. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

### 1. Prophylactic Methods

In one aspect, the invention provides a method for at least substantially preventing in a subject, a disease or condition associated with an aberrant expression or activity of a lupus-related protein, by administering to the subject an agent that modulates expression or at least one activity of the polypeptide. Subjects at risk for a disease that is caused or contributed to by aberrant expression or activity of a lupus-related protein can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

### 2. Therapeutic Methods

Another aspect of the invention pertains to methods of modulating expression or activity of lupus-related protein or gene for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. In another embodiment, the agent inhibits one or more of the biological activities of the lupus-related protein or gene. Examples of such inhibitory agents include antisense nucleic acid molecules and antibodies and other methods described herein. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a lupus-related polypeptide. In one embodiment, the method involves administering an agent (e.g., an agent identified by a screening assay described herein), or combination of agents that modulate (e.g., up-regulates or down-regulates) expression or activity. Inhibition of activity is desirable in situations in which activity or expression is abnormally high or up-regulated and/or in which decreased activity is likely to have a beneficial effect.

Photoprovocation Study

Using a stepwise permutation for assembling biomarker panels with the two different classifiers K-Nearest Neighbor (K-NN) and the Support Vector Machine (SVM), panels of these new marker peptides were successfully defined that predict the susceptibility to lupus (e.g., CLE), manifested in the development of a skin lesion after radiation or that reflect protection against such development with higher accuracy than each of the peptide markers alone. The combination of 2, 3 or 4 peptides clearly increased the performance of the marker panels by up to 20% as compared to the individual markers.

The classifiers aimed at defining panels that best fulfilled the two classification tasks—prediction and protection. The marker panel assemblage started with the testing of all possible 703 panels, i.e., all possible combinations of the 38 peptides as pairs. Those, which passed a predefined threshold, were accepted as suitable panels consisting of 2 different peptides. Subsequently, this process was repeated for 3- and 4-feature panels, considering 8,436 and 73,815 combinations of 3 and 4 different peptides, respectively. For each step, the area under the curve of the receiver operating characteristic curves (ROC-AUC) was calculated as measure of the performance of a panel.

The comparison of the ROC-AUC for the individual marker candidates with the data from the combinations of 2, 3 and 4 peptides showed a clear increase of these values with the size of the panels. The largest improvement of performance was obtained when assembling panels with 2 and 3 peptides. When a fourth peptide was added the ROC-AUC still increased but the increase was not as clear as in the preceding two steps. Since an increasing complexity of panels, i.e., increasing number of features (peptides) per panel, bears the risk of overfitting data, the panels from this study that were analyzed were restricted to a maximum of 4 peptides. This would represent a good combination of the performance of the panels and their complexity. Combinations of more than 4 peptides also form part of the profile of the present invention.

The use of two different classifiers allowed review of the panels that were found by the primary classifier K-NN. The majority of the 2-feature panels from K-NN were confirmed by SVM for prediction and protection demonstrating the robustness of the presented results. Based on the comparison of the results from both classifiers, the most frequent peptides within predictive panels were specific peptides deriving from alpha-1-antitrypsin, polymeric-immunoglobulin receptor, beta-defensin 1, collagen alpha-2(I) chain, hemoglobin alpha chain, fibrinogen alpha chain and serum albumin. For the scenario of protection against lesion the most frequent peptides in the 2-feature panels derived from beta-2-microglobulin, kininogen-1, polymeric-immunoglobulin receptor, complement C4-A, hemoglobin alpha chain, Ig kappa chainV-III and fibrinogen alpha chain. The selection of suitable marker panels is based on (1) the statistical results, (2) the role of a biomarker within the disease mechanism of lupus and (3) on the consideration if and how a marker can be included in an appropriate assay system.

The marker panels presented herein were retrieved by different bioinformatics tools appropriate for measuring disease response. The marker panels can be further validated experimentally in a subsequent step with relevant samples. An MS-based peptidomics analysis can provide a fast validation, a further specification and selection of the most suitable peptide panels that are presented in this report.

Furthermore, the assemblage of marker panels from previously detected marker peptides clearly increased the accuracy of prediction of lesion development and of protection against photoprovoked development of skin lesions in CLE patients from all disease subgroups. These marker panels can be used for developing assays for the use in further steps of this program. Such assays can be based on mass-spectrometric techniques, which had already been used for biomarker discovery, or on antibodies for immuno assays.

Multivariate Analysis Results for Biomarker Panel Classification: Prediction and Protection from Skin Lesion Development

A stepwise permutation for assembling Biomarker Panels for prediction and protection of lesion development was conducted using the K-Nearest Neighbor (K-NN) and the Support Vector Machine (SVM). Combinations of 2,3 and 4 peptides increased performance by up to 20% as compared to individual peptides. Based on the SVM and K-NN results, it is preferred to use no more than 4 peptide combinations for increasing marker performance. The following peptides (listed by precursor protein name) are recommended to be included in panels spanning from groups of 2 to 4:

### Predictive/Protective

PigR (SEQ ID NOS:23-25)
Hemoglobin—α chain (SEQ ID NO:35)
Fibrinogen—α chain (SEQ ID NOS:15 and 16)
Kininogen-1 (SEQ ID NOS:19-22)
C4-A (SEQ ID NOS:10-14)
Ig kappa chain V-III (SEQ ID NO:18)
β2-microglobulin (SEQ ID NO:5)

### Cell-Based Proteins:

The present invention enables the identification of biomarkers, e.g., cell surface markers, in CLE by using a human disease model and a biomarker tool that are complementary to each other: the application of photoprovocation in subjects diagnosed with CLE and a unique peptidomics platform that uses Mass Spectrometry and Projection to Latent Structures for multivariate data analysis for the identification of biomarkers.

Photoprovocation permits the development of skin lesions in a controlled and safe manner by exposing the subject to a personalized amount of triggering UVA/UVB radiation.

In this longitudinal study, data points from baseline to last patient visit can be correlated with confidence throughout the study. Taking into consideration that lesion development in CLE is seasonal and depends on both the intensity of ultraviolet radiation and the degree of the individual's sensitivity to light, photoprovocation can be used to trigger in a controlled way the disease in a cohort of subjects at the same time. Considering the high enzymatic activity in the dynamics of lupus, peptidomics yields stable molecules containing information about the disease process.

Coupling photoprovocation in CLE subjects with peptidomics is a novel strategy for the identification of biomarkers and lead peptides for drug development. The purpose of phototesting is two-fold: it first establishes the presence of a photosensitivity disorder and also reproduces the lupus lesions in CLE susceptible patients.

Plasma and skin biopsies from CLE subjects (divided into SOLE, DLE, LET) and healthy volunteers were collected at baseline and then again when either the skin lesion was developed or at the last patient visit if the lesion was not developed.

The present study provides a list of naturally occurring and novel peptides (8 from skin biopsies and 38 from plasma) that may be used as predictors of lesion development, and as monitors of drug response. The presence of these peptides in plasma suggests that circulating or systemic factors exist which perpetuate in CLE patients susceptible to UV-irradiation.

Some of these novel peptides may be useful as new molecular leads for the treatment of CLE (Protector peptides) and related inflammatory diseases where lymphocytes have to travel to the site of inflammation, including SLE.

The content of information in peptides in terms of indicating a disease state is higher than for genes or proteins, since peptides appear at the end of the metabolic pathway, subsumes genetic changes as well as changes in the enzymatic pathway or in protein expression. Furthermore, peptides are often characterized by increased half-life times and high permeability between tissue compartments. Therefore, peptides seem to be ideal biomarkers for CLE, with a high probability to reflect different pathologies and heterogeneity of CLE even beyond the site of the disease, e.g., in the blood circulation.

The features of the novel peptides are listed below:

### Use of Predictor Peptides

CLE subjects may be grouped at baseline as responders (develop lesions) or non-responders (do not develop lesions) before the use of photoprovocation. If a drug were being tested for the prevention of lesion development upon photoprovocation, the exclusion of non-responders from the study would decrease the number of false positive results in the active group and the placebo effect in the placebo group.
Predictor peptides provide insights about the mechanism of skin lesion development in CLE subjects in particular and tissue damage in SLE subjects in general. Therefore, these peptides may be useful to monitor drug response in clinical studies testing SLE treatments.
The scientific insight obtained from the predictor peptides may serve as a guide for the selection of new targets for the treatment of lupus.
The panel of peptides described as predictors, i.e., the presence of those panel members, may replace photoprovocation as a skin test for sensitivity to ultraviolet radiation. This statement becomes more relevant in SLE where photoprovocation has the potential for triggering flares.
Potential prediction of lesion development in DLE subjects based on the peptide derived from alpha-1-antitrypsin (M1 variant as opposed to the M2/M3 variant).

### Use of Protector Peptides

Protector peptides may also be useful in early clinical discovery when testing CLE or SLE treatments. The increase in plasma concentration of protector peptides may be an indication of positive drug response.
Protector peptides as markers of positive response to treatment may be a useful tool for physicians to evaluate whether or not the treatment of choice is adequate for the patient.
Either the protector peptides or the scientific information they provide may serve as targets or as guides for the selection of new targets for the treatment of lupus.
Protector peptides may be useful to define the lupus population that responds to a particular treatment.

### All Peptides

Unique source of information for data mining for the prediction/protection and mechanism of action for lesion development in CLE and potentially flares in SLE.

Because several disease variants exist and also a genetic involvement in CLE is very likely, panels based on the 38 peptides found in plasma were identified to cover the most prominent aspects of lupus pathophysiology. The combination of 2, 3 or 4 peptides clearly increased the performance of the marker panels by up to 20% as compared to the individual markers. With this method, the confidence of using some of the peptides as potential leads for new medications is also increased. These characteristics of panel peptides would make possible to recruit subjects with homogeneous baseline profiles since each peptide is connected with a particular disease factor or pathway.

### Study Subjects

Healthy volunteers and subjects with confirmed diagnosis by histological analysis of the following CLE subtypes were included:
a. Subacute Cutaneous Lupus Erythematosus (SOLE)
b. Discoid Lupus Erythematosus (DLE)
c. Lupus Erythematosus Tumidus (LET)

### UV Light Source

The light sources include a high-pressure metal halide lamp (323-436 nm) (Sellamed 3000, Sellas, Medizinische Gerate, Gevelsberg, Germany) for UVA phototesting and a UV-801 unit lamp with fluorescent bulbs (285-350 nm) Philipps TL 20 W/12 (Waldmann, Villingen/Schwenningen, Germany) for UVB phototesting. Irradiation output is monitored by means of a Variocontrol (Waldmann).

### UV Light Administration

### Minimal Erythema Dose, Immediate Pigment Darkening, and Minimal Tanning Dose

Minimal erythema dose (MED), threshold dose for immediate pigment darkening (IPD), and minimal tanning dose (MTD) are determined according to standard procedures. Using the Test Unit from Waldmann, defined areas of approximately 4.5 cm² on the lower back is irradiated with 6 different dosages of UVA (10, 20, 40, 60, 80 and 100 J/cm²) and 6 different dosages of UVB (25, 50, 75, 100, 125 and 150 mJ/cm²). Test reactions are read immediately (IPD) and 24 hours (MED and MTD) after irradiation.

### Provocative Phototesting

For provocative phototesting, areas (5×7 cm) of uninvolved skin on the upper back or extensor aspects of the arms are irradiated with the MTD for UVA (60-100 J/cm²) followed by 1.5 MEDs of UVB, daily for 3 consecutive days. Test areas are evaluated after each irradiation, 24 hours after the last irradiation and then weekly for up to 4 weeks. Healthy volunteers participating as control and subjects diagnosed with CLE follow the same phototesting procedures.

### Skin Biopsy Collection

In subjects diagnosed with CLE, one skin-punch biopsy (4 mm) is collected on the full-developed skin lesion, and one skin-punch biopsy (4 mm) at baseline. If the provocative phototesting does not induce skin lesions within 4 weeks from the last irradiation dose, skin-punch biopsy is collected from the irradiated site at the end of the 4-week.

A total of 2 skin-punch biopsies (4 mm) are also be collected from healthy volunteers, one at baseline and one on Week 2/Day 11.

### Blood Collection

Blood (5 mL) is collected at the same time points as described for skin biopsy collection. Plasma is separated from blood for peptidomics analysis.

### Analytical Methods

As measure for process quality check-up, each sample was spiked with a defined amount of an internal control (SOP-5046-02). After peptide extraction, samples were separated by liquid chromatography into 96 fractions and each fraction was subjected to mass spectrometry, using the ABI 4700 MALDI TOF-TOF platform (LIMS entries 113-060622-DPD03, 113-060622-DPD04, 113-060724-DPD05, 113-060818-DPD06), according to DBVN's SOP-5014 to -5051, SOP-5053 to -5067, SOP-5075-1 and SOP-5004. Mass spectra of all fractions were combined resulting in a two-dimensional display of peptide masses into a database for data analysis and statistical evaluation. Plasma samples were measured in duplicates in the MALDI mass spectrometer to enhance the sensitivity of the method for marker detection.

### Data Analysis

### Data Pre-Processing

The data pre-processing involved a base-line correction procedure, and m/z-recalibration of the mass spectrometric data. Then, the spectra were binned down to 1 Da resolution for data reduction and further processing steps and stored in a proprietary database. Adjacent spectra were used for confirmation of results, especially in cases where peptides eluted in more than one fraction. For the definition of master peptide displays the mass spectra of the same fraction from each experimental group were averaged. Also, master displays containing the standard deviation of the mass spectra were created. For the visualization of these mass spectrometric displays, a two-dimensional display view has been introduced. On the basis of the master peptide displays, peak detection served for the identification of 25,383 attributes in plasma and 13,864 attributes (e.g. peptides, variables) in skin. For statistical analysis and data reduction, the mass spectrometric intensities were stored into one data frame using the chromatographic fraction and mass spectrometric m/z ratio as labels for the attributes. The signal intensities of all attributes for each sample were normalized by quantile normalization and log transformed removing the effects of possible systematic sources of variation between batches and measurements.

### Outlier Analysis

Outlier samples were detected in a two step approach: initially a principal component analysis (PCA) was performed on the whole data frame as obtained for skin or plasma samples; subsequently, the multivariate Mahalanobis distances (Filzmoser et al. 2005) were computed using their scores in the first 5 principal components which were extracted by PCA. Multivariate outliers were defined as samples having a Mahalanobis distance exceeding the threshold of the 99.9% quantile of the chi-square distribution.

### General Strategy

The objective of this workpackage is to develop a predictive biomarker model for CLE disease flares. Univariate and multivariate data mining approaches were applied to identify biomarkers predicting lesion development after UV-radiation. Because the study cohort was split into two analytical sets with unbalanced experimental groups, a strategy was developed to correct for bias.

As outlined before, the two skin data sets were analyzed in joined model. The statistical power of the second plasma data set was increased by including more CLE subjects with developed lesions from the interim study group. A detailed description of the samples analyzed in the final study cohort is provided herein.

### Definition of Groups for Statistical Analysis

To make group comparisons, the different CLE subtypes were merged into two groups of “responders” and “non-responders” to UV-irradiation. The different groups were named as follows:
HV: Healthy volunteer
NR: Non-responder, no lesion developed after photoprovocation
R: Responder, lesion developed after photoprovocation
   In addition, samples before UV irradiation were referred to as “pre” and samples derived after to UV irradiation as “post.” The statistical design for the final analysis focused on these 6 groups, which were defined as response variables in the PLS modeling, or used in the multiple hypothesis testing. Specific peptide patterns for the following study questions were searched:
Predictive attributes: Searches for peptide patterns present in R group before photoprovocation and different to HV and NR.
Protective attributes: Searches for peptide patterns specific for the NR group before photoprocation and different from HV and R based on the assumption that protective mechanisms exist in NR.
Defensive mechanism attributes: Searches for peptide patterns specific for the NR group after photoprovocation and different from HV and R based on the assumption that counterregulatory mechanisms exist that prevent disease flares.
Lesion attributes: Searches peptide patterns present in the R group after photoprovocation and different from HV and NR.

### Multivariate Statistics: Multiple Response PLS Modeling

Multiple responses PLS modeling (Wold 2001) was used to identify biomarker candidates reflecting differences in their signal intensity among the experimental groups when analyzed simultaneously. The information from the experimental design was encoded into appropriate “response vectors,” created to express for each sample whether the condition belonging to one group was true or false. The variance associated with the process was also included using internal parameters. The orthogonal scores algorithm (Naes and Martens, 1989) was used to perform the PLS regression. Prior to the computation of the PLS model the X- and the Y-matrices were converted into Z-scores; hence, all variables were considered equally important. PLS computation was performed using the “pls” library in the statistical language R.

PLS regression coefficients were computed reflecting the significance of the relationship between the X-variables (attribute intensities) and the Y-vectors, and they were used for variable selection. In order to estimate the relevance of the multivariate regression coefficients, a permutation analysis was performed. A number of 750 PLS models were computed with the same data but randomized Y-vectors, and the null distribution of the regression coefficients was estimated. Attributes with regression coefficients higher than the defined threshold in at least one component and one group were included in the list of interest.

The results of the PLS modeling were visualized as bi-plots obtained by superimposing the PLS weights of the attributes and the Y-loadings of the response vectors. The bi-plots were generated for the most interesting projections found according to the experimental design, showing the relevance of the selected attributes. Within each bi-plot the significance increases as the distance from zero values increases.

PLS Modeling of Data from Skin Biopsies

PLS regression was performed to identify relationships between peptide intensities and the experimental groups. The projection technique reduces the dimensionality of multivariate data to embed the experimental variables and signals in a visualizable space. For each signal coordinate the distance to the origin indicates the variance in the reduced two-dimensional space. Signals without variance with respect to the study design would lie in the middle of the biplot. With PLS, linear combinations of the original experimental variables can thus be functionally interpreted. This enables a biological interpretation of the nature of coherent variation.

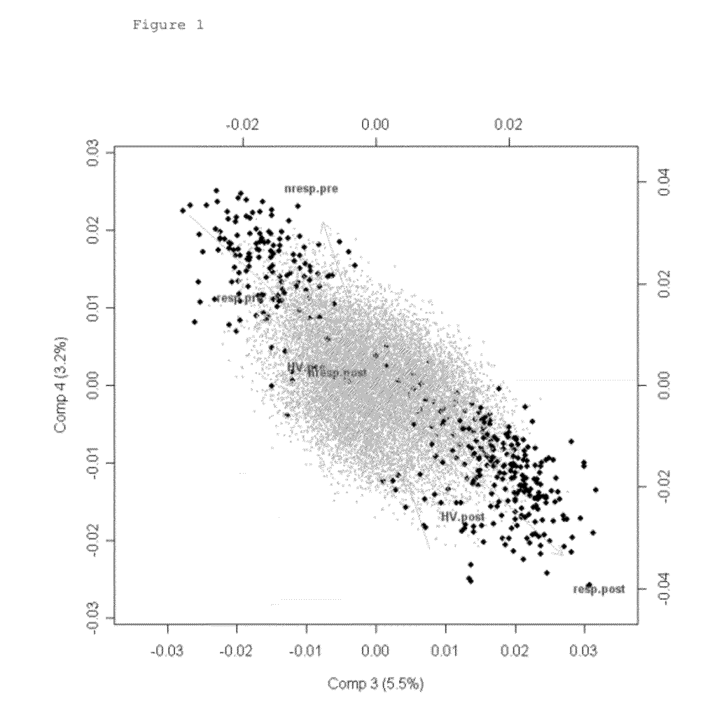

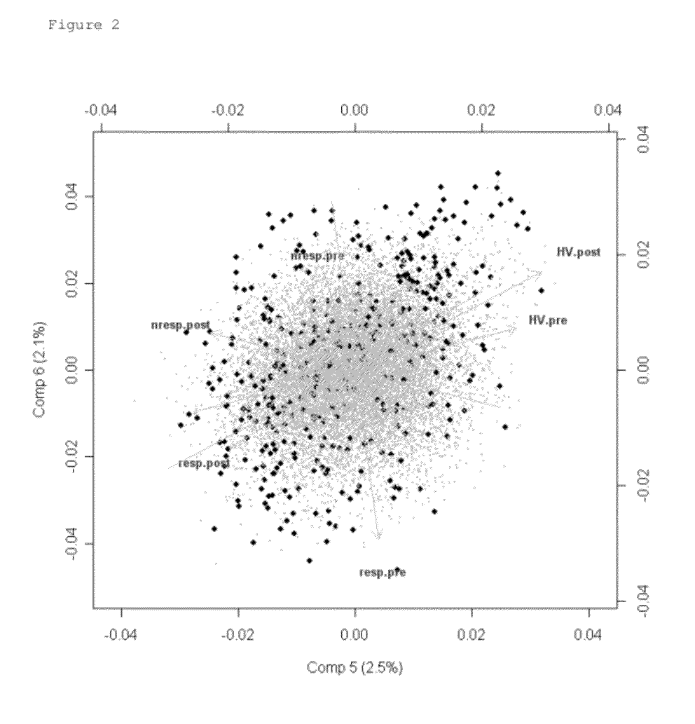

The first two PLS components capture pre-analytical and analytical process variance, whereas components 3 to 6 capture peptides responding to the experimental design. FIG. 1 and FIG. 2 summarize the most relevant projections using compoents 3 to 6. The biplot of components 3 and 4 (FIG. 1) approximates the effect of UV-irradiation on peptide patterns. As shown, substantial differences in peptide patterns between lesional and non-lesional skin of CLE responders to photoprovocation were found. These peptides either increased or decreased in lesional skin, and may thus be associated with the development of photoprovoked lesions. However, the plot shows that the cloud of signals is stretched along the Y-vector of the responder samples taken after photoprovocation. Signals correlating with the vector responder-post cannot be well separated from the vector HV-post, while the groups of the responders and non-responders before photoprovocation were in a reverse position. Thus, UV-irradiation and/or the longitudinal study design strongly impact the response pattern of all study groups.

The residual variance is described with the analysis of component 5 (FIG. 2), showing signals that discriminate HV (on the right) from responders or non-responders (on the left). Responders (above) and non-responders (below) could be further discriminated on component 6.

Selection of Peptides from Skin for Sequence Identification

Features (peptides) on the basis of the PLS model were obtained as follows: The regression coefficients were extracted from the PLS model for each attribute (e.g., peptide) against each response variable (i.e., the biological groups) for each component found to be mostly related to the experimental design (components 3, 4, 5, and 6). Signals exhibiting a significant regression coefficient (at p<0.05, or p<0.01, or p<0.001 of the corresponding null distribution obtained by permutation) were considered as relevant signals entering the list of interest. Features from the multiple hypothesis testing entered the list of interests on the basis of the corrected p-values. The number of relevant peptides which entered the list of interest is shown in Table 1.

TABLE 1
Number of relevant peptides detected by the
different tests at different significance levels

| | | p* < 0.05 | | p* < 0.01 | | p* < 0.001 | |
|---|---|---|---|---|---|---|---|
| | Ap- | De- | In- | De- | In- | De- | In- |
| Test | proach | creased | creased | creased | creased | creased | creased |
| R-pre | Multi- | 75 | 39 | 0 | 0 | 0 | 0 |
| | variate | | | | | | |
| | Uni- | 396 | 299 | 52 | 33 | 5 | 0 |
| | variate | | | | | | |
| R-post | Multi- | 178 | 268 | 6 | 22 | 0 | 0 |
| | variate | | | | | | |
| | Uni- | 517 | 507 | 163 | 85 | 39 | 3 |
| | variate | | | | | | |
| NR-pre | Multi- | 3 | 5 | 0 | 0 | 0 | 0 |
| | variate | | | | | | |
| | Uni- | 308 | 420 | 50 | 73 | 3 | 2 |
| | variate | | | | | | |
| NR-post | Multi- | 0 | 0 | 0 | 0 | 0 | 0 |
| | variate | | | | | | |
| | Uni- | 240 | 464 | 15 | 100 | 1 | 21 |
| | variate | | | | | | |
| Total | Multi- | 255 | 310 | 6 | 22 | 0 | 0 |
| (non- | variate | | | | | | |
| redun- | Uni- | 1214 | 1469 | 260 | 280 | 48 | 26 |
| dant) | variate | | | | | | |

(p* is the corrected p-value after 750 permutations)

In agreement with the description of the PLS model, most signals selected by the multivariate tests were either increased or decreased in the responder-post (R-post group). Compared to all other study groups the number of signals specific for the responder-pre group (R-pre) was highest indicating that this approach identified markers which distinguish between responders and non-responders to photoprovocation. The total number of unique signals (which is less then the mathematical sum) indicates the presence of redundant signals between the tests. For example, a marker specific for the responder group—pre is also specific post photoprovocation. Results obtained by the univariate approach are comparable (in ratios) to the ones obtained by PLS; the higher numbers observed are due to the assumption made in univariate testing where groups are considered independent from each other. In order to identify the most interesting peptides for subsequent identification (generation of the interesting lists for sequencing), an intersection of results from both approaches was created (Table 2).

TABLE 2
Summary of the candidates from skin at different significance levels.

| Candidates skin | p < 0.05 | p < 0.01 | p < 0.001 |
|---|---|---|---|
| Total number of unique peptides | 2800 | 542 | 74 |
| Intersection uni-/multivariate | 393 | 24 | 0 |

Total number of unique peptides: Number of signals that were found significantly differentiating between subgroups in the multi- OR the univariate approach.

Intersection uni-/multivariate: Number of signals that were found significantly differentiating between subgroups in the multi-AND the univariate approach.

Peptides were selected for sequence identification based on the list of 393 signals identified by multivariate analysis and in the univariate approach. Candidates were prioritized based on the rank statistics and their relevance in the PLS model.

PLS Modeling of Data from Plasma Samples

### PLS Modeling

A corresponding data mining approach was conducted for the plasma data set.

First, the most meaningful projections of the PLS model were identified by the analysis of the R² values obtained for every component and response. Components 3 to 6 explained most of the variance associated with the experimental design.

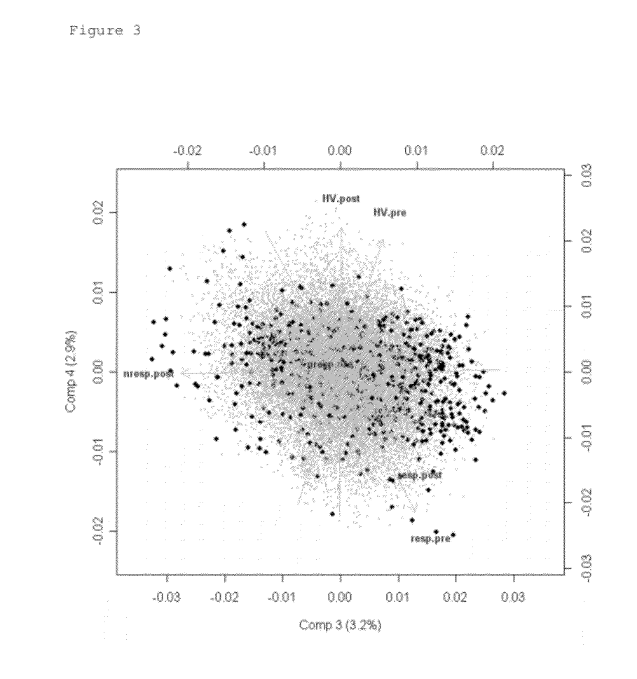

FIG. 3 shows a biplot of components 3 and 4. In this projection, no UV-radiation or pre/post sampling related effect in plasma samples was found for HV (in the upper section of the graph). However, the two components approximate the lesion development aspect of the data very well. Signals related to the group of responders (lower section of the graph) were clearly separated from the group of HV and non-responders. Interestingly, components 3 and 4 do not capture pre/post UV-radiation effects in the responder group. Moreover, the Y-vectors R-pre and R-post are high correlating with each other, suggesting that these markers are specific for responders to UV-irradiation and are associated with the development of lesions.

Components 5 and 6 capture markers that discriminate between responders and non-responders to UV-irradiation. Peptide signatures were found that distinguish responders and non-responders to photoprovocation in samples taken prior to UV-radiation. These circulating plasma markers already reflect differences between CLE patients with subclinical disease and may be useful to stratify CLE patients into responders and non-responders to photoprovocation.

Selection of Peptides from Plasma for Sequence Identification

The data from the multivariate analysis were combined with the results from the univariate analysis to create a list of interest:

TABLE 3
Number of relevant peptides detected by the
different tests at different significance levels

| | | p* < 0.05 | | p* < 0.01 | | p* < 0.001 | |
|---|---|---|---|---|---|---|---|
| | Ap- | De- | In- | De- | In- | De- | In- |
| Test | proach | creased | creased | creased | creased | creased | creased |
| R-pre | Multi- | 2 | 8 | 0 | 0 | 0 | 0 |
| | variate | | | | | | |
| | Uni- | 1695 | 1105 | 415 | 227 | 50 | 21 |
| | variate | | | | | | |
| R-post | Multi- | 4 | 21 | 0 | 0 | 0 | 0 |
| | variate | | | | | | |
| | Uni- | 1379 | 988 | 221 | 107 | 7 | 5 |
| | variate | | | | | | |
| NR-pre | Multi- | 349 | 290 | 22 | 13 | 0 | 0 |
| | variate | | | | | | |
| | Uni- | 1440 | 1229 | 314 | 193 | 45 | 20 |
| | variate | | | | | | |
| NR-post | Multi- | 2 | 9 | 0 | 0 | 0 | 0 |
| | variate | | | | | | |
| | Uni- | 2564 | 2451 | 815 | 810 | 108 | 145 |
| | variate | | | | | | |
| Total | Multi- | 354 | 321 | 22 | 13 | 0 | 0 |
| (non- | variate | | | | | | |
| redun- | Uni- | 4793 | 4238 | 1422 | 1163 | 193 | 184 |
| dant) | variate | | | | | | |

(p* is the corrected p-value after 750 permutations)

As already visualized by bipolar PLS plots, most of the signals selected by multivariate tests were related to the group responder pre, although some signals were also selected from the other groups. Also, in plasma the total number of unique signals indicates the presence of redundant signals, so responding significantly in more then one test.

The intersection of results from both approaches is shown in Table 4:

TABLE 4
Summary of the candidates from plasma at different significance levels.

| Candidates Plasma | p < 0.05 | p < 0.01 | p < 0.001 |
|---|---|---|---|
| Total number | 8977 | 2587 | 377 |
| Intersection | 593 | 31 | 0 |
| uni-/multivatiate | | | |

Total number of unique peptides: Number of signals that were found significantly differentiating between subgroups in the multi- OR the uni-variate approach.

Intersection uni-/multivariate: Number of signals that were found significantly differentiating between subgroups in the multi- AND the uni-variate approach.

A list of 593 signals was obtained from the intersection of the multivariate and univariate approach. For sequence identification, candidates were prioritized according to their statistical rank and their relevance in the PLS model. An additional 3 sequences were obtained from a preliminary analysis. These candidates were subjected to peptide identification ahead of the final statistical analysis.

### Peptide Identification

In total, 46 peptides were identified by means of tandem mass spectrometry (MS/MS) followed by product ion database searches (MASCOT search engine) and manual interpretation of the data.

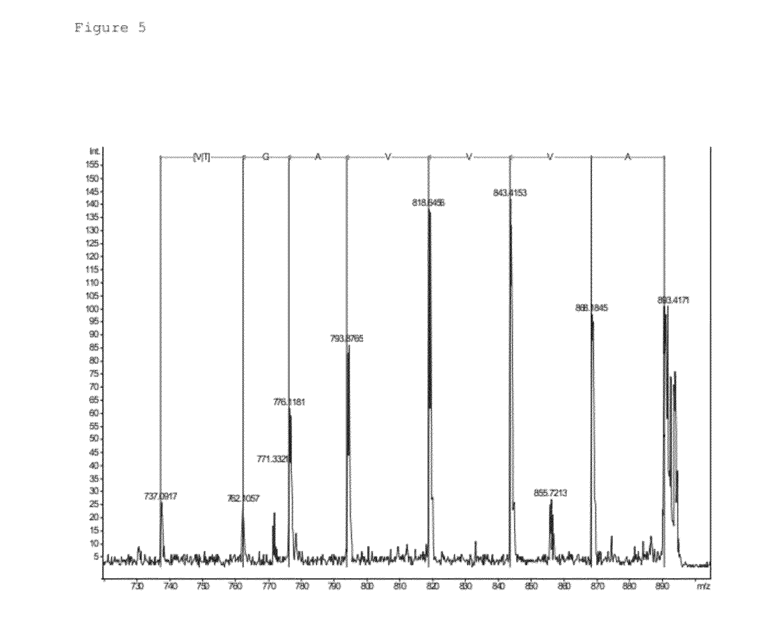

Because a non-invasive blood test is preferred over a skin biopsy for patient stratification, more resources were spent to identify plasma peptides compared to skin peptides. Nonetheless, peptides from skin were also identified, since they may mirror the underlying molecular processes and thus improve the understanding of the lupus pathology. FIG. 5 shows one example of one relevant peptide that was identified in plasma and derived from the immuno-molecule CD99 (SEQ ID NO:7).

Peptide Identification from Skin

From skin samples, 8 candidates were identified deriving from 8 different precursor molecules. Table 5 summarizes the identified peptides from skin. Shown are the candidates with fraction and their mass from the list of interest (IL-Mass) and the respective precursor.

TABLE 5
Results of peptide identification from skin

| | | | | | | | Novel |
|---|---|---|---|---|---|---|---|
| | | | | | Type of | | disease |
| Fr. | IL mass | AA | Sequence | Precursor Name | biomarker | Regulation | peptide |
| 79 | 11729.5 | 21-119 | (SGLEA) IQRTPKIQVY | Beta-2-microglobulin | Lesion | ↑ R | Known |
| | | | SRHPAENGKS NFLNCYVSGF | | | | |
| | | | HPSDIEVDLL | | | | |
| | | | KNGERIEKVE HSDLSFSKDW | | | | |
| | | | SFYLLYYTEF TPTEKDEYAC | | | | |
| | | | RVNHVTLSQP KIVKWDRDM | | | | |
| 49 | 2877.5 | 139-165 | (SHKER)FD ESGKGKGIAG | Protein CGI-38 | Lesion | ↓ R | New |
| | | | RQDILDDSGY VSAYK(NAGTY) | | | | |
| 71 | 1677.5/3354 | 999-1028 | (YDVPY)GESHLFRVPSYQ | Collagen alpha-1(VI) | Predictive | ↓ R | New |
| | | | ALLRGVFHQTVSRKVALG | chain | | | |
| 39 | 3524.5 | 592-624 | (TSYNR)GDSTFESKS | Fibrinogen alpha chain | Predictive* | ↓ R | New |
| | | | YKMADEAGSE ADHEGTHSTK | | | | |
| | | | RGHA(KSRPV) | | | | |
| 35 | 1989.5 | 27-43 | (TQEKN)TLPT | Thymosin bata-10 | Radiation | ↑ | New |
| | | | KETIEQEKRS EIS | | response | | |
| 42 | 3108.6 | 1-28 | SDAAVDTSSE ITTKDLKEKK | Prothymosin alpha | Radiation | ↑ | New |
| | | | EVVEEAEN(GRDAP) | | response | | |
| 48 | 1822.5/3644 | 24-58 | (DTYRR)SAVPPGADKKAEAGL | Ribosomal protein S10 | Radiation | ↑ | New |
| | | | GQQPEFQFRGGFGRGRGQPP | | response | | |
| 29 | 2503.5 | 1097-1123 | (LSWGK)GSSG GSGAKPSDAA | Eukaryotic translation | Radiation | ↑ | New |
| | | | SEAARPATST LNR(FSALQ) | intiation factor 4 | response* | | |
| | | | | gamma 1 | | | |
| | | | | (elF-4-gamma 1) | | | |

*indicates that a peptide was only found in the PLS model.

The rows of the table describe the fraction of the candidate in a peptide display (Fr.), its molecular mass (IL mass), the direction of change within a comparison, the accession number and the precursor name used in the SwissProt database (http://www.expasy.org), the position of the first and last amino acid of the sequenced peptide within the precursor molecule (aa-position), the precursor name, the amino acid sequence (preceding and following amino acids are given in brackets) and possible comments.

The eight peptides identified were classified regarding their response behavior to photoprovocation and their ability to classify the study groups (Table 5). The main categories are:
Predictive biomarker: A peptide that is present prior to UV radiation and which predicts lesion development in CLE responder subjects; Collagen alpha-1(VI) chain) and Fibrinogen alpha chain.
Lesion biomarkers: A peptide correlating positively or negatively with disease exacerbation/lesion development; Beta-2-microglobulin (SEQ ID NO:5) and CGI-38 (SEQ ID NO:42).
Radiation biomarkers: A peptide indicating a response to UV photodamage in all subjects;

Thymosin beta-10 (TYB10), Prothymosin alpha (PTMA), Ribosomal protein S10 (S10), and Eukaryotic translation initiation factor 4 gamma 1 (IF4G1) (SEQ ID NO:42).

Five protein precursors can be classified as primarily intracellular proteins (TYB10, PTMA, IF4G1 and S10) and three protein precursors (collagen, fibrinogen and beta-2-microglobulin) as extracellular proteins or components of the extracellular matrix (ECM).

In some cases (PTMA and TYB10), these intracellular proteins may also have a functional role in the extracellular compartment. PTMA and TYPB10, which belong to the thymosin alpha and beta family, are also implicated in cell proliferation, cell migration and wound repair.

One peptide is a fragment of IF4G1 a translational initiating factor, which stabilizes AU-rich mRNA, which encodes proteins in apoptosis and wound repair.

Another peptide is a fragment of the ribosomal protein S10, for which SLE patients frequently develop autoantibodies.

Another identified peptide is a fragment of CGI-38 with unknown cellular function.

Some of these peptides were also increased in HV (TYB10, PTMA, IF4G1 and S10) after photoprovocation, indicating that UV-exposure causes inflammatory and proliferative cellular responses.

One peptide (Beta 2 Microglobulin) represents a secreted protein. It is up-regulated after radiation in samples from responders.

A C-terminal fragment from collagen VI was found to be down-regulated at both time points in samples from responders. This peptide may allow a prediction of the development of a lesion as response to UV radiation.

Three of the identified peptides were found to be significant in a first analysis but failed in the subsequent selection based on the more strict selection of candidates with data from multi- and univariate analyses. Here, they did not fulfill the significance level of 0.05 in the univariate testing. These peptides were identified from the precursors IF4G1, fibrinogen alpha-chain and the ribosomal protein S10.

The main findings in skin are summarized in Table 5 and a description of the members of the skin panel is below.

### Beta-2-Microglobulin (B2MG HUMAN) (SEQ ID NO:5)

Precursor description: Beta-2-microglobulin (B2MG, SwissProt accession no. P61769) has a molecular mass of approximately 12. B2MG is a protein associated with the light chain of HLA antigens, expressed on the surface of antigen presenting cells and found in low concentrations in body fluids. B2MG is also an essential component of the neonatal Fc receptor (FcRn), which plays a critical role in regulating IgG homeostasis in vivo.

Peptide description: The identified peptide spans 99 amino acids at positions 21-119 covering the complete B2MG molecule.

Peptide level: The protein is up-regulated in samples from responders after photoprovocation. B2MG had also been identified from skin. The significance of the findings in the context of LE is discussed in the chapter on peptides identified in plasma.

Biomarker type: Lesion

Disease context: IFN-γ upregulates the expression of B2MG which is required for cell surface MHC class I expression. Increased B2MG levels may thus reflect increased IFN-γ levels in skin and increased antigen presentation. B2MG is also a subunit of the neonatal Fc Receptor (FcRn) which regulates the transport of IgG through epithelia (Yoshida et al., 2004; Kobayashi et al.2002). The FcRn receptor is also expressed on moncytes and dendritic cells (Zhu et al. 2001). High levels of B2MG have been described in autoimmune disease, such as SLE, rheumatoid arthritis, Sjogren's syndrome and Crohn's disease. Thus, increased expression of B2MG in lesions may be marker of disease activity.

F079.11729.5

(SEQ ID NO: 5)

(SGLEA) IQRTPKIQVY SRHPAENGKS NFLNCYVSGF
HPSDIEVDLL KNGERIEKVE HSDLSFSKDW SFYLLYYTEF
TPTEKDEYAC RVNHVTLSQP KIVKWDRDM(—)

Sequence of identified peptide from B2MG above. The identified peptide from the C-terminus of the precursor is depicted in bold; the amino acids adjacent to its N-terminus within the precursor molecule are given in brackets.

### Collagen Alpha-1(VI) Chain [Precursor] (SEQ ID NO:39)

Precursor description: The collagen alpha-1(VI) chain precursor (acc. no. P12109) is unusual among collagens due to the small size of its collagenous domains and in its supramolecular structure. It has been called “short-chain collagen.” It is relatively resistant to bacterial collagenase and has a glycine content less than one-third of the protein, suggesting interrupted helical regions. Electron microscopy shows additional unique features. Collagen VI is a component of microfibrillar structures in many tissues. These microfibrils localize close to cells, nerves, blood vessels, and large collagen fibrils and are considered to have an anchoring function. Consistent with such a function are the biochemical findings that type VI collagen binds cells and that its fusion protein binds type I collagen. The binding activity also implies that, in addition to a structural role, type VI collagen may be involved in cell migration and differentiation and embryonic development. Additionally Type VI collagen also serves as a binding site for von Willebrand Factor (vWF) in the vascular subendothelium, where the type VI collagen-vWF complex may play an important role modulating the hemostatic response to vascular injury.

Description of the peptide: The peptide (aa 999-1028) is a fragment of the C-terminal global domain (aa 593-1028) of Collagen alpha-1(VI) chain.

Peptide levels: The peptide is down-regulated at both time points in samples from responders.

Biomarker type: Predictive

Disease context: The peptide has the potential for prediction of development of a lesion prior to photo-provocation. INF-γ down-regulates the expression of Collagen type VI.

Soluble collagen VI can rescue cells from apoptosis. A biological implication of these putative finding results from evidence in literature pointing to the inhibition of apoptotic cell death by collagen VI or peptides derived from the precursor molecule [RUHL et al, 1999].

Since the number of apoptotic cells is increased in skin lesions after UV-radiation (KUHN et al, 2005) a direct link to the pathogenesis is given. It has been shown that the expression of collagen type VI is down-regulated by INF-γ (Heckmann et al. 1989) suggesting that INF-γ levels are increased before lesion development.

Amino acid sequence of the identified CO6A1 peptide above. The identified peptide from the C-terminus of the precursor is depicted in bold; the amino acids adjacent to its N-terminus within the precursor molecule are given in brackets.

### Eukaryotic Translation Initiation Factor 4 Gamma 1 (IF4G1 HUMAN) (SEQ ID NO:40)

Synonymes: eIF-4-gamma 1, eIF-4G1, eIF-4G 1, p220

Precursor description: The IF4G1 precursor (acc. no. Q04637) has a molecular mass of 175,535 spanning 1,600 amino acids. It is a translational initiating factor involved in recruitment of mRNA for translation: All eukaryotic cellular messenger RNAs are posttranscriptionally modified by addition of an m(7)GTP moiety to the 5-prime terminus, referred to as a cap. Recognition of the cap structure and unwinding of mRNA secondary structure during the initiation phase of protein synthesis is catalyzed by initiation factors of the eIF4 group. Collectively, these factors facilitate the recruitment of mRNA to the ribosome, which is the rate-limiting step for protein synthesis under normal conditions (Haghighat et al. 1997, Lindquist et al. 2000).

Peptide description: The identified peptide spans 27 amino acids at positions 1097-1123.

Peptide levels: The level of the peptide is increased after photoprovocation. Also, it appears that the level of the peptide is lower in CLE responders before and after photoprovocation.

Disease context: During apoptosis protein synthesis can be down-regulated by degradation of eIF4G1. Polypeptide chain initiation factor eIF4GI undergoes caspase-mediated degradation during apoptosis to give characteristic fragments (Bushell 2000). The most prominent of these has an estimated mass of approximately 76 kDa (Middle-Fragment of Apoptotic cleavage of eIF4G; M-FAG). The identified peptide is released from the C-terminus eIFGI. Although only found in the PLS model, the peptide appears to increase after UV radiation in all study groups.

F029.2503.5
(SEQ ID NO: 40)
(...LSWGK)GSSG GSGAKPSDAA SEAARPATST LNR(FSALQ...)

Sequence of identified peptide from IF4G1 above. The identified peptide is depicted in bold; the amino acids adjacent to its N- and C-terminus within the precursor molecule are given in brackets.

### Fibrinogen Alpha Chain (FIBA HUMAN) (SEQ ID NO:41)

Precursor description: Fibrinogen is a 340-kDa protein that is found predominantly in plasma but is also bound to the surface of circulating platelets. It is composed of two sets of three non-identical chains connected by disulfide bonds, resulting in a tri-nodular structure with one central domain, and two identical outer domains. The C-terminus (aa 220-610) of fibrinogen alpha chain precursor (acc. no. P02671), also referred to as the C domain, represents a rich source of structural markers for probing the various functionalities associated with fibrinogen's role in maintaining hemostasis. This region is involved in fibrin polymerization; it serves as a substrate for Factor XIIIa and plasmin and binds to endothelial cells.

Peptide description: The identified peptide is a proteolytical fragment of the fibrinogen alpha chain (aa 592-624). The cleavage at basic amino acids suggests involvement of plasmin or kallikrein.

Peptide levels: The level of the peptide is lower in CLE responders before photoprovocation and is increased during lesion development. The signal intensity was low across all analyzed samples.

Disease context: Fibrinogen has an important role in tissue repair by providing an initial matrix that can stabilize wound fields and support local cell proliferation and migration (Geer et al. 2003, Koolwikj et al. 2003, Drew et al. 2001). Fibrin and fibrinogen is degraded by tissue plasmin and reduced activation of plasminogen to plasmin impairs wound healing (Romer et al. 1996). Decreased levels of fibrinopeptides indicate reduced ability in wound healing. Fibrinogen alpha expression is induced by IL-6 (Hu et al. 1995).

F039.3524.5

(SEQ ID NO: 41)

(...TSYN R)GDSTFESKS YKMADEAGSE ADHEGTHSTK
RGHA(KSRPV...)

Sequence of identified peptide from the fibrinogen alpha chain above. The identified peptide is depicted in bold; the amino acids adjacent to its N- and C-terminus within the precursor molecule are given in brackets.

### Protein CGI-38 (CG38 HUMAN) (SEQ ID NO:42)

Precursor description: The protein CGI-38 is a 176 aa protein (acc. no. Q9BW30, molecular mass 18,985) belonging to the p25 family. Sequence analysis demonstrates that p25 belongs to the highly conserved p25 gene family present in mammals, flies, nematodes, and even tetrahymenae. The human genome contains at least three p25-like genes, designated as p25-alpha, p25-beta, and cgi-50 (acc. nos O94811; P59282 and Q9BW30).

CGI-38 has been described as a brain specific protein. Its detection also in skin as described in this project is explained by the fact that skin as well brain ontogentically originate from the same germinal sheet (ectoderm).

Peptide description: The identified peptide spans 27 amino acids at positions 139-165.

Peptide levels: In CLE responders the level of the peptide is decreased during lesion development. The signal was not detectable in samples from center 010.

Biomarker type: Protective factor

Disease context: The function of the protein is unknown.

F049.2877.5

(SEQ ID NO: 42)

(...SHKER)FD ESGKGKGIAG RQDILDDSGY
VSAYK(NAGTY D...)

Sequence of identified peptide from CGI-38 above. The identified peptide is depicted in bold; the amino acids adjacent to its N- and C-terminus within the precursor molecule are given in brackets.

### Prothymosin Alpha (PTMA HUMAN) (SEQ ID NO:43)

Precursor description: The prothymosin-alpha (PTMA) precursor is a 110-aa protein (acc. no. P06454, molecular mass 12072). It is highly acidic with 54 out of 111 residues carrying an acidic moiety. In the thymus gland several hormones or hormone-like substances are produced from PTMA. The first 28 amino acids of the precursor constitute thymosin-alpha-1 that was originally isolated from calf thymosin fraction 5 and was shown to restore various aspects of immune function in several in-vitro and in-vivo test systems. Thymosin-alpha-1, is generated, at least partially, by the lysosomal asparaginyl endopeptidase legumain.

Peptide description: The identified peptide comprises the first 28 amino acids of the N-terminus (position 1-28) and represents the complete thymosin-alpha-1 molecule.

Peptide levels: The peptide is increased in HV and CLE responders after UV radiation.

Biomarker type: Radiation response

Disease context: Prothymosin alpha is an extremely abundant nuclear oncoprotein-transcription factor essential for cell cycle progression and proliferation that has been recently suggested as an anti- apoptotic factor (Letsas et al. 2006). Prothymosin alpha is processed to a naturally occurring peptide thymosin alpha 1 by the lysosomal asparaginyl endopeptidase legumain (Sarandeses et al 2003). Legumain is a key protease in class-II MHC antigen processing. The proteolysis of prothymosin in lymphocytes and other cells may suggest that thymosin alpha 1 has some biological function.

In the form of a synthetic 28-amino acid peptide, thymosin alpha 1 is in clinical trials worldwide for the treatment of some viral infections, malignancies, and HIV/AIDS. The mechanism of action of the synthetic polypeptide is not completely understood, but it is thought to be related to its immunomodulating activities on T-cells. In vitro experiments have shown that thymosin alpha 1 also activates dendritic cells to express MHC class II molecules (Huang et al. 2004; Romani et al. 2006).

F042.3108.5

(SEQ ID NO: 43)

(—)SDAAVDTSSE ITTKDLKEKK EVVEEAEN(GR DAP...)

Sequence of identified peptide from PTMA/thymosin-alpha-1 above. The identified peptide is depicted in bold; the amino acids adjacent to its C-terminus within the precursor molecule are given in brackets.

### Thymosin Beta-10 (TYB10 HUMAN) (SEQ ID NO:44)

Precursor description: A peptide from thymosin beta-10 (acc. no. P63313, 43 aa in total, molecular mass of 4,894) was identified. The precursor peptide belongs to the family of beta-thymosins (Hannappel et al. 2003), Huff et al. 2001). These are related peptides that were initially isolated from calf thymus and have been found afterwards in a wide variety of mammalian cells and tissues.

Peptide description: The identified peptide spans the 17 C-terminal amino acids of positions 27-43.

Peptide levels: The peptide reveals a response pattern similar to PTMA.

Biomarker type: Radiation response

Disease context: The release of a peptide from an intracellular protein may be associated with cell damage or associated to an immune-modulatory event (e.g. leukocytes infiltration). TYB10 peptides have recently been described in wounded skin (Huang et al. 2006). Overexpression of TYB10 in cancer cells leads to apoptosis (Lee et al. 2005). TYB10 inhibits cell migration and inhibits angiogenesis (Mu et al. 20069. TYB10 levels are increased following UV radiation independently of disease.

F035.1989.5

(SEQ ID NO: 44)

(...TQEKN)TLPT KETIEQEKRS EIS(—)

Sequence of identified peptide from TYB10 above. The identified peptide is depicted in bold; the amino acids adjacent to its N-terminus within the precursor molecule are given in brackets.

### Ribosomal Protein S10 (Q14489 Human) (SEQ ID NO:45)

Precursor description: Identified was a protein from a fragment of the 40S ribosomal protein S10 that is listed in SwissProt with the accession number Q14489 (58 amino acids, molecular mass 6281). This particular fragment has a high homology to the 40S ribosomal protein S10 itself (P45783, 165 amino acids, molecular mass 18898), but with distinct differences.

The small ribosomal subunit protein S10 is involved in Escherichia coli in binding tRNA to the ribosome, and also operates as a transcriptional elongation factor.

Peptide description: The identified peptide spans the 33 C-terminal amino acids at positions 24-58.

Peptide levels: The level of the peptide is increased in CLE patients after UV-radiation. The signal was not detectable in samples from center 010. The reason for this observation cannot be elucidated with the available data.

Biomarker type: Radiation response

Disease context: Autantibodies reacting with ribosomal protein S10 (anti-S10) have been described in patients with systemic lupus erythematosus (SLE), and also in SLE mouse models (Anderson et al. 2001; Bonfa et al. 1989). Patients who had both anti-Sm and anti-S10 antibodies showed lower serum complements levels, high frequency of skin lesion and anti-double-stranded DNA antibody. Many anti-Sm antibodies may recognize B/B′, D, and S10 simultaneously, and such antibodies may appear in active disease (Hasegawa et al. 1999). The peptide contains the carboxyl-terminal Gly-Arg-Gly region of S10 protein which is involved in constructing the anti-Sm cross-reactive epitope (Hasegawa et al. 1998). The identified peptide is increased following UV radiation and may thus represent an autoantigen in SLE/CLE.

F048.1822.5
(SEQ ID NO: 45)
(...DTYRR)SAVPPGADKKAEAGLGQQPEFQFRGGFGRGRGQPP(—)

Sequence of identified peptide from Ribosomal protein S10 above. The identified peptide is depicted in bold; the amino acids adjacent to its N-terminus within the precursor molecule are given in brackets.

TABLE 6
Potential involvement of identified peptides and
protein precursors in CLE lesion development

| | Type of | Regu- | | Cyto- | |
|---|---|---|---|---|---|
| Precursor Name | biomarker | lation | IL-6 | kines | CLE |
| Beta-2- | Lesion | ↑ R | no | INF-β, | IgG deposition |
| microglobulin | | | effect | INF-γ | in tissue, |
| | | | | | antigen |
| | | | | | presentation |
| Protein CGI-38 | Lesion | ↓ R | un- | | |
| | | | known | | |
| Collagen alpha- | Predictive | ↓ R | un- | INF-γ | Decreased |
| 1(VI) chain | | | known | | levels may |
| | | | | | indicate |
| | | | | | impairements |
| | | | | | in skin to |
| | | | | | escape |
| | | | | | apoptosis |
| Fibrinogen | Predictive* | ↓ R | IL-6 | | Abnormalities |
| alpha chain | | | | | in fibrinolysis. |
| Eukaryotic | Radiation | ↑ | un- | alter | UV radiation |
| translation | response* | | known | stability | induces eIFG |
| | | | | | degradation, |
| | | | | | inhibits cap- |
| | | | | | dependent |
| | | | | | protein |
| | | | | | synthesis and |
| | | | | | induces |
| | | | | | apoptosis |
| Thymosin beta- | Radiation | ↑ | un- | | inhibitor of |
| 10 | response | | known | | angiogenesis |
| | | | | | and tumor |
| | | | | | growth |
| Prothymosin | Radiation | ↑ | un- | | immuno- |
| alpha | response | | known | | modulatory |
| | | | | | effects, |
| | | | | | promotes |
| | | | | | CD4- |
| | | | | | expressed DC |
| | | | | | differentiation, |
| | | | | | suppress the |
| | | | | | up-regulated |
| | | | | | IL-12 |
| | | | | | production |
| Ribosomal | Radiation | ↑ | un- | | Patients with |
| protein S10 | response | | known | | anti-S10 |
| | | | | | antibodies |
| | | | | | showed lower |
| | | | | | serum |
| | | | | | complements |
| | | | | | levels, high |
| | | | | | frequency of |
| | | | | | skin lesion |

*indicates that a peptide was only found in the PLS model

Peptide Identification from Plasma

38 peptides from plasma were identified deriving from 20 different precursor proteins. Table 7 lists the identified peptides. Given are the identifier of the peptides (fraction and mass) and their amino acid sequence and information regarding their potential involvement in CLE/SLE. Peptides were classified according to their response behavior to photoprovocation and their ability to classify the study groups. The main categories are:
Predictive biomarker: A peptide that is present prior to UV radiation and which predicts lesion development in CLE responder subjects
Lesion biomarkers: A peptide correlating positively or negatively with disease exacerbation/lesion development
Protective biomarkers: A peptide which is different in non-responder CLE patients compared to responder CLE patients indicating biological mechanisms that protect or counteract (defensive) lesion development.

To interpret the findings in the context of the autoimmune diseases cutaneous and/or systemic LE, Pubmed was quiried for coocurrences of the protein name and SLE or CLE. Whenever found, information regarding involvement of IL-6 in the regulation of gene expression was provided. To correlate the response behavior to photoprovocation with biological pathways, protein precursors were grouped as follows:
Immune relevant proteins
Complement
Protease inhibitors
Collagens
Fibrinogen
Kininogen
Miscellaenous

TABLE 7
Table 7: Identified plasma peptides

| | | | | Type of | | |
|---|---|---|---|---|---|---|
| Fr. | IL mass | Sequence | Precursor Name | biomarker | Regulation | |
| | | | | | | Novel |
| | | | | | | disease |
| | | | | | | peptide |
| 79 | 11730.5 | (SGLEA) IQRTPKIQVY SRHPAENGKS NFLNCYVSGF HPSDIEVDLL KNGERIEKVE | Beta-2-microglobulin | Predictive | ↑ R | Known |
| | | HSDLSFSKDW SFYLLYYTEF TPTEKDEYAC RVNHVTLSQP KIVKWDRDM(—) | | | | |
| 54 | 1964/3927 | (LGHRS)DHYNCVSSGGQCLYSACPIFTKIQGTCYRGKAKCCK | Beta-defensin 1 | Predictive | ↑ R | New |
| 62 | 3560.5 | (DLADG)VSGGEGKGGSDG GGSHRKEGEE ADAPGVIPGI VGAVVVA(VAGAI) | CD99 antigen | Predictive | ↑ R | New |
| 79 | 11774.5 | EIVLTQSPGTLSLSPGERATLSCRASQSVSNSYLAWYQQKPGQAPRLLIYG | Ig kappa chain | Predictive | ↑ R | New |
| | | ASSRATGIPDRFSGSGSGTDFTLTISRLEPDDFAVYYCQQYGSSPQTFGQ | | | | |
| | | GSKVEIKR | | | | |
| 55 | 3834.5 | (FAEEK)AVADTRDQADGSRASVDSGSSEEQGGSSRALVSTLVPLG(LVLAV) | Polymeric IgR | Predictive | ↑ R | New |
| 60 | 5233.5 | (EIENK)AIQ DPRLFAEEKA VADTRDQADG SRASVDSGSS EEQGGSSRAL | Polymeric IgR | Predictive | ↑ R | New |
| | | VSTLVPLG(LVLAV) | | | | |
| 46 | 4352.5 | (EIENK)AIQDPRLFAEEKAVADTRDQADGSRASVDSGSSEEQGGSSRA(LVSTL) | Polymeric IgR | Predictive | ↑ R | New |
| 47 | 1968.5 | (LPSRS)SKITHRIHWESASLLR(SEETK) | Complement C3 | Lesion | ↑ R post | New |
| 36 | 1499.5 | (SSTGR)NGFK SHALQLNNR(QIRGL) | Complement C4-A | Protective | ↑ NR post | New |
| 46 | 1896.5 | (SSTGR)NGFKSHALQLNNRQIR(GLEEE) | Complement C4-A | Protective | ↑ NR post | New |
| 42 | 1879.5 | (SSTGR)NGFKSHALQLNNRQIR(GLEEE) | Complement C4-A | Protective | ↑ NR post | New |
| 33 | 1362.5 | (NGFKS)HALQLNNRQIR(GLEEE) | Complement C4-A | Protective | ↑ NR post | New |
| 33 | 1449.5 | (RNGFK)SHALQLNNRQIR(GLEEE) | Complement C4-A | Protective | ↑ NR post | New |
| 42 | 6687.5 | (FSPFR)S SRIGEIKEETTVSPPHTSMA PAQDEERDSG KEQGHTRRHD | Kininogen-1 | Protective | ↑ NR post | New |
| | | WGHEKQ(RKHN L) | | | | |
| 20 | 1980.5 | (HGHQR)GHG LGHGHEQQHG LGHGHK(FKLDD) | Kininogen-1 | Protective | ↑ NR post | New |
| 48 | 1076.5 | (ISLMK)RPPGFSPFR(SSRIG) | Kininogen-1 | Protective | ↑ NR post | New |
| 20 | 1923.5 | (GHQRG)HGLGHGHEQQHGLGHGHK(FKLDD) | Kininogen-1 | Protective | ↑ NR post | New |
| 80 | 4546.5 | (AMFLE)AI PMSIPPEVKF NKPFVFLMIEQNTKSPLFMG KVVNPTQK(—) | Alpha-1-antitrypsin | Polymorphism/ | ↑ R | New |
| | | | | Predictive | | |
| 77 | 2067.5/2067.5 | (EAI PM)SIPPEVKF NKPFVFLMIE QNTKSPLFMG KVVNPTQK(—) | Alpha-1-antitrypsin | Polymorphism/ | ↑ R | New |
| | | | | Predictive | | |
| 38 | 1154.5 | (PVSAM)EPLGRQLTSGP(NQEQV) | Alpha-2-antiplasmin | Predictive* | ↑ R | New |
| | | | | | | New in |
| | | | | | | SLE/CLE |
| 24 | 2659.5 | (SYKMA)DEAGSE ADHEGTHSTK RGHAKSRPV(R DCDD) | Fibrinogen alpha chain | Predictive | ↑ R | New |
| 39 | 2769.5 | (PSRGK)SSSYS KQFTSSTSYN RGDSTFESKS(YKMAD) | Fibrinogen alpha chain | Predictive* | ↑ R | New |
| 51 | 4591.5 | (FLVKS)QGVNDNEEGFFSARGHRPLDKKREEAPSLRPAPPPISGGGYR(A | Fibrinogen beta chain | Lesion | ↓ R | New |
| | | RPAK) | | | | |
| 42 | 1611.5 | (GAKGA)N GAPGIAGAPG FPGARGP(SGP QG) | Collagen alpha-1(I) chain | Predictive | ↓ R | New |
| 43 | 2481.5 | (GEPGP)TGLPGPPGERGGPGSRGFPGADGVAGP(KGPAG) | Collagen alpha-1(I) chain | Predictive | ↓ R | New |
| 26 | 1938.5 | (FPGLP)GPSGEPGKQGPSGASGERGPPGP(MGPPG) | Collagen alpha-1(I) chain | Predictive* | ↓ R | New |
| 29 | 2940.5 | (GPMGP)R GPPGPPGKNG DDGEAGKPGR PGERGPPGP(QGARG) | Collagen alpha-1(I) chain | Predictive | ↓ R | New |
| 29 | 2353.5 | (PGPPG)TSGH PGSPGSPGYQ GPPGEPGQAG P(SGPPG) | Collagen alpha-1(III) chain | Predictive | ↓ R | New |
| 33 | 3025.5 | (PGAAG)ARGNDGARGSDGQPGPPGPPGTAGFPGSPGAKG(EVGPA) | Collagen alpha-1(III) chain | Predictive | ↓ R | New |
| 34 | 2825.5 | (AGAPG)LRGGAGPPGPEGGKGAAGPPGPPGAAGTPGLQG(MPGER) | Collagen alpha-1(III) chain | Lesion | ↓ R | New |
| 21 | 1192.5 | (GPSGR)DGLPGPPGSPGPP(GQPGY) | Collagen alpha-1(IV) chain | Lesion | ↓ R | New |
| 26 | 2443.5 | (KGQKG)EPAIIEPGMLIEGPPGPEGPAGLPG(PPGTM) | Collagen alpha-1(V) chain | Predictive* | ↑ R | New |
| 26 | 2823.5 | (NGLDG)LKGQPGAPGVKGEPGAPGENGTPGQTGARG(LPGER) | Collagen alpha-2(I) chain | Predictive | ↓ R | New |
| 55 | 1505.5 | (AQGTP)DVSSALDKLKEFGN(TLEDK) | Apolipoprotein C-I | Protective | ↑ NR post | New |
| 54 | 1699.5 | (HASLD)KFLASVSTVLTSKYR | Hemoglobin alpha chain | Predictive | ↑ R | New |
| 40 | 1434.5 | (QIKKQ)TALVELVKHKPKA(TKEQL) | Serum albumin | Predictive | ↓ R | New |
| 43 | 2357.5 | (GVFRR)DAHKSE VAHRFKDLGE ENFK(ALVLI) | Serum albumin | Predictive | ↑ R | New |
| 55 | 3324.5 | (HPGLR)AAPGQEPPEHMAELQRNEQEQPLGQWHLS(KRDTG) | Sulfhydryl oxidase 1 | Lesion* | ↓ R post | New |

The identified peptide is depicted; the amino acids adjacent to its N-terminus within the precursor molecule are given in brackets.

The table shows the identified peptides from plasma. Given are the candidates with fraction and their mass from the list of interest (IL-Mass), mode of change (⇑=increase or ⇓=decrease in R=responder or NR non-responder group), SwissProt name (http://www.expasy.org), position of the first and last amino acid of the sequenced peptide (sequence range), precursor name, amino acid sequence with the preceding and following amino acids and possible comments. * peptide was found by PLS, only

Immune-Relevant Precursor Proteins and Peptides

CD99 Antigen [Precursor] (SEQ ID NO:7)

Synonyms: T-cell surface glycoprotein E2, E2 antigen, Protein MIC2, 12E7

Precursor description: CD99 (acc. no. P14209) is a surface molecule that is present on thymocytes, T cells, many other hematopoietic cell types and endothelial cells. It has been implicated in a number of cell-cell adhesion and cell-activation phenomena. Ligation of CD99 on activated and memory T cells stimulates and induces their adhesion to VCAM-1-expressing cell monolayers. CD99 engagement on human peripheral blood T cells results in TCR/CD3-dependent cellular activation and allows for Th1-restricted cytokine production (Waclavicek et al. 1998).

Peptide description: The identified peptide spans the C-terminus of the extracellular region and part of the transmembrane region. One explanation is that CD99 is shed from the plasma membrane of CD99 positive cells.

Peptide levels: The level of the CD99 peptide is increased in CLE patients who develop a lesion before and after photoprovocation compared to controls.

Biomarker type: Predictive

Disease context: CD99 is broadly expressed on lymphocytes and endothelia cells and plays a major role in leukocyte transmigration (Bixel et al. 2004, Schenkel et al. 2002; Imbert et al. 2006). In a mouse model for cutaneous delayed-type hypersensitivity (DTH) reaction, anti-CD99 antibodies inhibited the recruitment of in vivo-activated T cells into inflamed skin as well as edema formation. It was concluded that mouse CD99 participates in the in the transendothelial migration (TEM) of lymphocytes and in their recruitment to inflamed skin in vivo. This establishes CD99 as a valid target for interference with cutaneous inflammatory processes. Upregulation and/or increased shedding of CD99 may increase the rate of transendothelial migration of immune cells into skin. Shedding of CD99 may be required to release the attached cell from the endothelium.

TABLE 8

| Key | From aa | to aa | Length | Description |
|---|---|---|---|---|
| SIGNAL | 1 | 22 | 22 | |
| CHAIN | 23 | 185 | 163 | CD99 antigen. |
| TOPO_DOM | 23 | 122 | 100 | Extracellular (Potential). |
| TRANSMEM | 123 | 147 | 25 | Potential. |
| TOPO_DOM | 148 | 185 | 38 | Cytoplasmic (Potential). |
| VAR_SEQ | 159 | 185 | | (in isoform II) |

TABLE 9
Alignment of the CD99 peptide against the sequence of the precursor

```
                        CD99_HUMAN
MARGAALALLLFGLLGVLVAAPDGGFDLSDALPDNENKKPTAIPKKPSAGDDFDLGDAVV
                        F062.3560.5 -----------------------------------------
-------------------
                        CD99_HUMAN
DGENDDPRPPNPPKPMPNPNPNHPSSSGSFSDADLADGVSGGEGKGGSDGGGSHRKEGEE
                        F062.3560.5 --------------------------------------
VSGGEGKGGSDGGGSHRKEGEE
                                  Transmembrane Region
                        CD99_HUMAN
ADAPGVIPGIVGAVVVAVAGAISSFIAYQKKKLCFKENAEQGEVDMESHRNANAEPAVQR
                        F062.3560.5 ADAPGVIPGIVGAVVVA------------------------
-------------------
CD99_HUMAN TLLEK
F062.3560.5 -----
Underlined: extracellular domain (aa 122-100),
Italics: transmembrane region (aa 147-125)
```

Polymeric-Immunoglobulin Receptor [Precursor] (SEQ ID NOS:23-25)

Synonyms: Poly-Ig receptor, PIGR, Hepatocellular carcinoma-associated protein TB6, contains secretory component.

Precursor description: The polymeric Ig receptor (plgR, acc. no. P01833), also called membrane secretory component (SC), mediates epithelial transcytosis of polymeric immunoglobulins (plgs). J Chain-containing polymeric IgA (plgA) and pentameric IgM bind plgR at the basolateral epithelial surface. After transcytosis, the extracellular portion of the plgR is cleaved at the apical side, either complexed with plgs as bound SC or unoccupied as free SC. The plgR receptor is expressed on several glandular epithelia including those of liver and breast (Kaetzel et al. 2005).

Peptide description: The three identity peptides (aa 598-639 (SEQ ID NO:23), 598-648 (SEQ ID NO:24), 610-648 (SEQ ID NO:25)) span an unstructured region that links the Ig-like domain 5 to the transmembrane region and a short part of the transmembrane region. The mechanism by which plgR is cleaved to SC and the precise cleavage site are currently unknown. Studies using free SC purified from colostrums showed that processing can occur on multiple cleavage sites and is likely to be cell-type specific. Here, we found two major cleavage products that predict cleavage to occur at Lys598 or Lys610 (Asano et al. 2004).

Peptide levels: All three identified peptides are increased in CLE patients that develop a lesion before and after photoprovocation compared to controls.

Biomarker type: Predictive

Disease context: PIgR is the rate limiting component of IgA and IgM transport in mucosa, epithelia and lung. The plgR also highly expressed in liver (Seilles et al 1995). The region N-terminal to the identified peptides corresponds to the secretory component of plgR which is released after transport of IgA and IgM. The identified peptide may thus reflect the rate of IgA and IgM transport into tissue, mucosa and lung (Kontos et al. 2005). The pro-inflammatory cytokines INF-γ, TNF-α, and IL-1, which are produced in response to infection and inflammation, play a key role in upregulation of plgR expression. It may thus be concluded that CLE responder to UV-irradation have elevated levels of pro-inflammatory cytokines before disease flares occur.

Expression of plgR in keratinocytes has also been reported (Nihei et al. 1995, Nihei et al. 1996). It has been suggested that SC plays an antiinflammatory role in the pathogenesis of inflammatory skin diseases via inhibition in keratinocytes of IFNγ induced expression of ICAM-1 and HLA-DR. Since IgM appears to be the predominant individual Ig in CLE, it may be concluded that the SC compound also stabilizes IgM.

TABLE 10
Alignment of PigR peptides against the sequence of the precursor

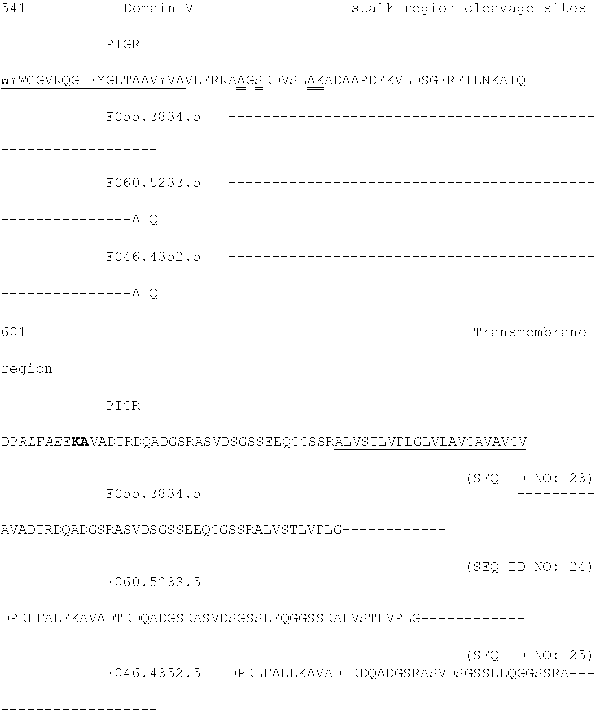

Italics: proposed cleavage site, bold: identified cleavage site, double underlined: ragged C-terminus

Beta-2-Microglobulin (SEQ ID NO:5)

Peptide levels: In contrast to skin biopsies the peptide is already increased in CLE patients who develop a lesion before photoprovocation.

Biomarker type: Predictive

Disease context: B2MG is the non-covalently bound light chain of the human class I major histocompatibility complex (MHC-I) and the beta-chain of the neonatal Fc Receptor (FcRn), a 45-kD chain closely related to MHC class FcRn recycles immunoglobulin (Ig) G from most cells and transports it bi-directionally across epithelial barriers to affect systemic and mucosal immunity. Recent studies have shown that FcRn rescues IgG from intracellular lysosomal degradation by recycling it from the sorting endosome to the cell surface (Junghans et al. 1996). The FcRn is functionally expressed in monocytes, intestinal macrophages, and dendritic cells (Zhu et al. 2001). Mice deficient in either FcRn or B2MG have an abnormally short serum half-live of IgG (Israel et al. 1996; Ghetie et al. 1996; Christianson et al. 1997). It was found that the absence of B2MG or FcRn protects mice against an autoantibody-mediated disease, resembling SLE (bullous pemphigoid) (Liu et al. 1997; Akilesh et al. 2004). These data suggest that increased plasma levels of B2MG reflect increased stability and/or transport of IgG in lesional CLE.

### Ig Kappa Chain V-III Region SIE (SEQ ID NO:18)

Precursor description: The amino acid sequence of the Ig kappa chain V-III region SIE was first described by Andrews and Capra in 1981 (acc. no. P01620, 109 amino acids, 11,775 Kda). It was derived from an idiotypically related human monoclonal rheumatoid factor and was obtained by Edman degradation of the intact light chains. The published sequence is a proteolytic fragment and comprises the Ig kappa chain variable region III.

Peptide description: The peptide described here was also identified by Edman degradation and corresponds to the 109 amino acids of the complete precursor molecule.

Peptide levels: The level of the kappa light chain fragment is increased both in CLE patients who develop a lesion before photoprovocation and those that do not develop a lesion compared to healthy controls.

Biomarker type: Predictive

Disease context: Free light chains (FLC) are a natural product of B lymphocytes and, as such, represent a quantifiable biomarker of cellular proliferation (Hopper et al. 2000). Accurate measurement of the concentrations of these components in serum and urine provides a unique means of ascertaining B cell immunoglobulin synthesis during physiologic and especially pathologic states, where such information has important diagnostic and therapeutic implications (van der Heijden et al 2006). Elevated levels of free light chains occur in primary amyloidosis, multipe myeloma, lymphocytic neoplasms, Waldenströms macroglobulinaemia and connective tissue diseases such as SLE (Hopper et al. 1989; Redegeld et al. 2002). Sensitive assays are available to measure free light chains in urine and serum (Bradwell et al. 2001). The identified peptide corresponds to the kappaV-III idiotype, which has been described as the predominantly autoantibody idiotype produced by patients with rheumatoid arthritis and SLE (Newkirk et al. 1993). This observation is now confirmed for CLE patients in the current project where elevated plasma levels of this peptide were found in CLE patients.

### Beta-Defensin 1 [Precursor] (SEQ ID NO:6)

Synonyms: BD-1, Defensin, beta 1, hBD-1

Precursor description: Defensins comprise a subclass of small, cysteine-rich, cationic antimicrobial peptides produced by higher organisms. Mammalian defensins are further classified into α-defensins and β-defensins based on both precursor and gene structure, as well as a pattern of six cysteines forming three disulfide bonds and an overall length of 25-45 amino acids. Additionally, alpha-defensins are found in neutrophils as well as Paneth cells of the small intestine, whereas human β-defensins (hBD) are products of epithelial tissues. To date, six hBDs, hBD-1 through to -6, have been identified in human tissues. hBD-1, the precursor of the here identified peptide marker, was first described at the Lower Saxony Institute for Peptide Research (IPF) in human blood filtrate and later in urine (acc. no. P60022) (Bench et al 1995; Zucht et al. 1998). It is constitutively produced by various epithelial tissues, including urogenital and respiratory tracts and skin.

Peptide description: The identified peptide (F054.1964/3927) corresponds to the mature form of hBD-1, i.e., it comprises the 36 amino acids that are released from the precursor after truncation of signal and propeptide.

Peptide levels: The peptide is increased in CLE patients who develop a lesion (responder) compared to non-responder CLE patients.

Biomarker type: Predictive

Disease context: The mRNA of hBD-1 is expressed in keratinocytes, monocytes, monocyte-derived-macrophages (MDM), and monocyte-derived-dendritic cells (DC) (Sorensen et al. 2005; Niyonsaba 2005; Harder et al. 2005; Supp et al. 2004). hBD-1 mRNA expression by monocytes and MDM was increased after activation with IFN- and/or lipopolysaccharide (LPS) in a dose- and time-dependent fashion (Ryan et al. 2003). Expression of hBD-1 mRNA by immature DC was low, and increased considerably after maturation. While the precise function of hBD-1 is currently unknown, increased plasma levels of hBD-1 in CLE patients who will develop a lesion after photoprovocation suggest activation of antigen presenting cells by INF-y before disease flare occur.

(SEQ ID NO: 6)

| | |
|---|---|
| 1 | [symbol] [symbol] [symbol] [symbol] [symbol] GNFLTGLGH |
| | RSDHYNCVSS GGQCLYSACP IFTKIQGTCY |
| 60 | |
| 61 | RGKAKCCK |

Alignment of hBD-1 against the sequence of the precursor above. The italic letters refer to the signal peptide (aa 1-21), bold to the propeptide (aa 22-32) and the sequenced peptide is shown underlined (aa 33-68).

The following peptides originate from precursor proteins having a role in the immune system. Available information on induction of gene expression is listed. Interestingly, a strong association with INF-γ was found. Table 11 summarizes how the identified peptides may be related to the CLE/SLE disease pathology.

TABLE 11
Peptides from immune-relevant precursor
proteins and their relation to CLE/SLE

| Pre- | Type | | | | | |
|---|---|---|---|---|---|---|
| cursor | of bio- | Regu- | | Cyto- | | |
| Name | marker | lation | IL-6 | kines | CLE | SLE |
| Beta-2- | Pre- | ↑ R | no | INF-β, | IgG | Marker of |
| micro- | dictive | | effect | INF-γ | deposition | disease |
| globulin | | | | | in tissue, | activity |
| | | | | | antigen | |
| | | | | | presentation | |
| Beta- | Pre- | ↑ R | no data | INF-γ | Anti- | Anti- |
| defensin | dictive | | found | | microbial, | microbial, |
| 1 | | | | | apoptotic, | apoptotic, |
| | | | | | expressed in | expressed in |
| | | | | | skin and | skin and |
| | | | | | immune | immune |
| | | | | | cells | cells |
| CD99 | Pre- | ↑ R | no data | | Major role | Major role |
| antigen | dictive | | found | | in the | in the |
| | | | | | leukocyte | leukocyte |
| | | | | | diapedesis, | diapedesis, |
| | | | | | induces | induces |
| | | | | | Th1-type | Th1-type |
| | | | | | cytokine | cytokine |
| | | | | | production | production |
| | | | | | (TNF-α, | (TNF-α, |
| | | | | | INF-γ) | INF-γ) |
| lg kappa | Pre- | ↑ R | in- | | free light | free light |
| chain | dictive | | creased | | chains as a | chains as a |
| | | | | | marker of | marker of |
| | | | | | persistant B- | persistant B- |
| | | | | | cell activity | cell activity |
| Poly- | Pre- | ↑ R | no data | INF-γ, | IgA and IgM | Glomerulo- |
| meric | dictive | | found | TNF-α, | deposition in | nephritis, |
| IgR | | | | IL-1, | tissues, | lupus |
| | | | | IL-4, | Complement | nephritis |
| | | | | sex hor- | activation | |
| | | | | mones | | |

### Complement Factors

The following paragraph describes peptides derived from the complement system identified in plasma samples. The complement system has long been known to be activated in exacerbations of SLE, particularly reflecting nephritic activity. It has been debated whether this complement activation is important in the pathogenesis of SLE or whether it is an innocent epiphenomenon. The literature on complement and SLE is conflicting (Walport 2002). Complement involvement in CLE is not as common as in SLE. Compared to SLE much less is known about systemic complement involvement in CLE, but it appears to be less common as in SLE.

### Complement C4-A [Precursor] (SEQ ID NO:10-14)

Precursor description: Complement C4 (acc. no. P0C0L4) is expressed in the liver and to a lesser extent in immune cells. C4 plays a central role in the activation of the classical pathway of the complement system. Prior to secretion, the single-chain precursor is proteolytically cleaved to yield the non-identical chains alpha, beta and gamma. During activation, the alpha chain is cleaved by activated C1 into the anaphylotoxin C4a (77 aa) and the 690-aa protein C4b-A. The alpha chain fragment C4b-A stays linked to the beta and gamma chains. It is the major activation product and is an essential subunit of the C3 convertase (C4b2a) and the C5 convertase (C3bC4b2a) of the classical complement pathway. Further degradation of C4b-A by C1 into the inactive fragments C4c-A and C4d-A blocks the generation of C3 convertase. The expression of C4 is regulated by INF-γ.

Peptide description: Five peptides were identified from the complement C4 precursor. All originate from the complement C4 alpha chain from the region aa 1137-1352. The N-terminus of three of these peptides maps to the C4d-A cleavage site in the alpha chain fragment C4b-A. The peptides covering the amino acids 1337-1349 and 1337-1352 have already been described (Villanueva et al., 2006).

Peptide levels: The level of all five peptides increased in non-responders under photoprovocation.

Biomarker type: Protective, defensive

Disease context: Disturbance in the clearance of apoptotic cells is considered one of the potential pathophysiological mechanisms underlying breakdown of tolerance and, subsequently, the induction of SLE and CLE (Kuhn et al. 2006). It was shown that reduced uptake of apoptotic cells by macrophages in SLE correlates with decreased serum levels of complement (Bilj et al. 2006)

The identified peptide is the C-terminal fragment of the C4b-A cleavage. The N-terminal fragment is C4d-A, which is deposited on normal erythrocytes, while abnormal levels have been observed on the surface of erythrocytes of patients with systemic lupus erythematosus (SLE). It was proposed that C4d-bound to reticulocytes and/or platelets may serve as biomarkers of disease activity in patients with SLE (Liu et al. 2005; Manzi et al. 2004; Navratil et al. 2006). While C4d-bound to platelets was positively associated with disease activity and negatively associated with plasma C4 levels, we found that the corresponding soluble peptide is increased in non-lesional CLE patients after photoprovocation. The findings of this study suggest that higher levels of C4 breakdown products fascilitate clearance of apoptotic cells and protect non-responder CLE patients from developing lesions.

TABLE 12
Alignment of the C4b-A peptides against the sequence of the precursor

```
                        P0C0L4
WIETTAYALLHLLLHEGKAEMADQASAWLTRQGSFQGGFRSTQDTVIALDALSAYWIASH
                        F033.1449.5 ------------------------------------------
------------------(SEQ ID NO: 14)
                        F033.1362.5 ------------------------------------------
------------------(SEQ ID NO: 13)
                        F036.1499.5 ------------------------------------------
------------------(SEQ ID NO: 10)
                        F042.1879.5 ------------------------------------------
------------------(SEQ ID NO: 12)
                        F046.1896.5 ------------------------------------------
------------------(SEQ ID NO: 11)
                        P0C0L4
TTEERGLNVTLSSTGRNGFKSHALQLNNRQIRGLEEELQFSLGSKINVKVGGNSKGTLKV
                        F033.1449.5 --------------------SHALQLNNRQIR----------
------------------(SEQ ID NO: 14)
                        F033.1362.5 ---------------------HALQLNNRQIR----------
------------------(SEQ ID NO: 13)
F036.1499.5 ----------------NGFKSHALQLNNR-------------------------
------(SEQ ID NO: 12)
                        F042.1879.5 ----------------NGFKSHALQLNNRQIR----------
------------------(SEQ ID NO: 11)
                        F046.1896.5 ----------------NGFKSHALQLNNRQIR----------
------------------(SEQ ID NO: 11)
Underlined is the sequence of the C4d-A section of the Complement 4 alpha chain
```

### Complement C3 [Precursor] (SEQ ID NO :9)

Precursor description: Complement C3 (acc. no. P01024) plays a central role in the activation of the complement system. Its expression is regulated by IL-6 (Wilson et al. 1990). Its processing by C3 convertase is the central reaction in both classical and alternative complement pathways. After activation C3b can bind covalently, via its reactive thioester, to cell surface carbohydrates or immune aggregates. C3b is rapidly split in two positions by factor I and a cofactor to form iC3b (inactivated C3b) and C3f. Thus, C3f is released during the inactivation of the active complement form C3b. Subsequently iC3b itself is slowly cleaved (possibly by factor I) to form C3c and C3dg. Other proteases catalyze further breakdown resulting in fragments, such as C3d or C3g.

Peptide description: The identified peptide (F047.1968.5) constitutes the C3f peptide shortened by one amino acid at the N-terminus. C3f is a heptadeca peptide liberated during the catabolic degradation of C3b in blood. Ganu et al. (1989) suggested that the C3f peptide functionally resembles C3a anaphylatoxin and found that C3f is a weak spasmogen.

Biomarker type: Lesion

Peptide levels: The level of C3f fragment is increased in CLE patients developing a lesion after photoprovocation. However, compared to healthy subjects the peptide shows no clear regulation, which can be explained a supposed breakdown of C3f and the lability of the breakdown products.

Disease context: Numerous studies investigated the role of complement in the pathogenesis of SLE. It is commonly held that the complement system is activated during periods of active SLE (Manzi et al, 1996; Negi et al. 200; Rother et al. 1993). However, difficulties measuring complement activation products arise, because of the short half-life of these products. It has been suggested that C3 degradation products may also also reflect C3 activation, but are more stable and easier to analyze.

One of the important physiological functions of the classical pathway of complement activation is the clearance of circulating immune complexes (Walport 2002). Cleavage of C3b releases C3f and C3bi, which binds complement receptors involved in clearance of apoptotic cells. C3bi is also involved in maintaining B-cell tolerance (Sohn et al. 2003). Whether C3f levels are an indirect correlate of C3bi levels remains to be determined.

TABLE 13
Alignment of the C3f peptide against the sequence of the precursor

```
                        CO3_HUMAN
RLKGPLLNKFLTTAKDKNRWEDPGKQLYNVEATSYALLALLQLKDFDFVPPVVRWLNEQR
                        F047.1968.5 ------------------------------------------
------------------
                        CO3_HUMAN
YYGGGYGSTQATFMVFQALAQYQKDAPDHQELNLDVSLQLPSR [symbol]  SKITHRIHWESASLL [symbol]
                        F047.1968.5 ------------------------------------------
--SKITHRIHWESASLLR
                        CO3_HUMAN
SEETKENEGFTVTAEGKGQGTLSVVTMYHAKAKDQLTCNKFDLKVTIKPAPETEKRPQDA
                        F047.1968.5 ------------------------------------------
------------------
Underlined is the C3dg (955-1303) section of the complement C3. The C3f peptide (aa 1304-1320) (SEQ ID NO: 9) is highlighted in italics. Bold: factor I cleavage sites (R/S) aa 1303-1304 and 1320-1321 releasing the C3f fragment.
```

Coagulation

Changes in the coagulation system and a prothrombin state are common in SLE and are associated with a high prevalence of cardiovascular disease due to accelerated atherosclerosis, as well as an increased risk of venous thromboembolisms (Afeltra et al. 2004; Inoh et al. 1996). It has long been recognized that a molecular crosstalk exists between the coagulation and inflammation pathways. In particular, IL-6, plays a key role in in the associations between systemic inflammation and abnormalties in the coagulation system (Spronk et al. 2004).

### Fibrinogen B [Precursor] (SEQ ID NO:17)

Precursor description: Fibrinogen (acc. no. P02675) has a double function: yielding monomers that polymerize into fibrin and acting as a cofactor in platelet aggregation. The expression of Fibrinogen B is induced by IL-6 (Huber et al. 1990; Dalmon et al. 1993).

Peptide description: The identified peptide (31-72) is an N-terminal fragment of the fibrinogen B (FGB) chain and comprises the fibrinogen B activation peptide (FibB) as well as the first 28 amino acids of the mature beta chain.

Peptide levels: The peptide (F051.4591.5) is decreased in CLE patients who develop a lesion (responder to photoprovocation).

Biomarker type: Lesion

Disease context: The regulation of the peptides indicates abnormalities in coagulation and/or fibrinolysis in CLE responders to photoprovation. These abnormalities manifest during lesion development.

TABLE 14
Alignment of the identified fibrinogen peptide against the
sequence of the precursor.

```
                        FIBB_Human
MKRMVSWSFHKLKTMKHLLLLLLCVFLVKSQGVNDNEEGFFSARGHRPLDKKREEAPSLR
                        F051.4591.5 ------------------------------
QGVNDNEEGFFSARGHRPLDKKREEAPSLR
                        FIBB_Human
PAPPPISGGGYRARPAKAAATQKKVERKAPDAGGCLHADPDLGVLCPTGCQLQEALLQQE
                        F051.4591.5 PAPPPISGGGYR------------------------------
------------------
The activation peptide is highlighted in bold (aa 31-44).
```

### Fibrinogen Alpha Chain [Precursor] (SEQ ID NOS:15 and 16)

Precursor protein: Fibrinogen (acc. no. P02671) has a double function: yielding monomers that polymerize into fibrin and acting as a cofactor in platelet aggregation.

Peptide description: Two peptides (F024.2659.5, F039.2769.5) were identified from the fibrinogen alpha chain (FGA aa 605-629 and 576-600, respectively). The peptides are likely produced during fibrinolysis. The peptides covering the amino acids 576-600 has also been described by Villanueva et al. (2006).

Peptide levels: The level of both peptides is increased in CLE patients who develop a lesion.

Biomarker type: Predictive

Disease context: The two identified FGA peptides are likely generated by fibrinolysis. The regulation of the peptides suggests that CLE patients who develop a lesion have coagulatory and fibrinolytic disturbances. The fibrinolytic system is closely linked to control of inflammation, and plays a role in disease states associated with inflammation. Plasmin, in addition to lysing fibrin clots, also cleaves the complement system component C3, and fibrin degradation products have some vascular permeability inducing effects (Castellino and Ploplis, 2005).

TABLE 15
Alignment of the identified fibrinogen peptide A against the
sequence of the precursor

FIBA_HUMAN
VSETESRGSESGIFTNTKESSSHHPGIAEFPSRGKSSSYSKQFTSSTSYNRGDSTFESKS
F024.2659.5 ------------------------------------------------------
------
F039.2769.5 -----------------------------------
SSSYSKQFTSSTSYNRGDSTFESKS
(SEQ ID NO: 16)
FIBA_HUMAN
YKMADEAGSEADHEGTHSTKRGHAKSRPVRDCDDVLQTHPSGTQSGIFNIKLPGSSKIFS
F024.2659.5 ----DEAGSEADHEGTHSTKRGHAKSRPV-------------------------
------
(SEQ ID NO: 15)
F039.2769.5 ------------------------------------------
------------------

### Protease Inhibitors

The following peptides were found derived from protease inhibitors. Alpha-2-plasmin inhibitor and alpha-1 antitrypsin play an important role in controlling the activity of two major serine proteases plasmin and neutrophil elastase, respectively. A imbalance of the protease inhibitors and proteases is associated with thrombosis and tissue distruction.

### Alpha-2-Antiplasmin [Precursor] (SEQ ID NO:3)

Synonyms: Alpha-2-plasmin inhibitor, Alpha-2-PI, Alpha-2-AP

Precursor description: Alpha-2-AP (acc. no. P08697) is plasma glycoprotein that is a member of the SERPIN family of proteinase inhibitors. Alpha-2-AP is the primary fast-acting inhibitor of plasmin in vivo, but has also been reported to inhibit other enzymes such as trypsin, elastase, and activated Protein C (Coughlin et al. 2005). Alpha-2-AP is expressed by the liver and secreted in plasma. Structural and kinetic studies showed that Alpha-2-AP has three functional sites: a plasminogen/plasmin binding site, a reactive site that binds covalently the catalytic serine residue of plasmin, and a cross-linking site to the fibrin chain. Alpha-2-AP is abundant in plasma, where it exerts its antifibrinolytic properties by competing with fibrin for plasminogen binding through plasmin inhibition. The functional importance of Alpha-2-AP is illustrated by the rare reported cases of congenital Alpha-2-AP deficiency, which exhibits severe lifelong hemorrhagic tendency (Matsuno 2006).

Peptide description: The peptide corresponds to the proposed propeptide of alpha-2AP (aa 29-39), but lacks the methionine at position 28. It therefore is a marker for the cleavage rate of alpha-2AP. There are two N-terminal forms of Alpha-2-AP that circulate in human plasma: a 464-residue protein (Met-2AP) that includes the proposed propeptide and a 452-residue version (Asn-2AP) that lacks the propeptide. The N-terminal 12-residue peptide of Met-2AP was reported to be cleaved in the circulation by a soluble form of the fibroblast activation protein (FAP), a cell-surface proteinase that promotes invasiveness of certain epithelial cancers. In normal human plasma, the ratio of Met-2AP to the shorter Asn-2AP was estimated as about 30% to 70% (Lee et al. 2006).

Peptide levels: The level of the peptide is increased in CLE patients who develop a lesion after photoprovocation (responders). The peptide was found in the PLS model, only.

Biomarker type: Lesion

Disease context: The identified propeptide of alpha-2-AP marks the conversion of Met-2AP into the more easily fibrin-incorporable form, Asn-2AP. It was suggested that this step is associated with an increase in plasmin inhibition. Patients with SLE have an increased risk of thrombosis, related to the lupus anticoagulant or anticardiolipin antibodies and reduced fibrinolysis (Kawakami et al. 1992). Taken together with changes found in the level of fibrinogen alpha and beta peptides, these finding suggest disturbances in the coagulation and fibrinolysis system in CLE patients with developed lesions.

TABLE 16
Alignment of the alpha-2-antiplasmin peptide against the
sequence of the precursor

```
                                                                                                            PROPEP
                        A2AP_HUMAN (SEQ ID NO: 3)
MALLWGLLVLSWSCLQGPCSVFSPVSAMEPLGRQLTSGPNQEQVSPLTLLKLGNQEPGGQ
                        F038.1154.5 ----------------------------EPLGRQLTSGP---
------------------
```

### Alpha-1-Antitrypsin [Precursor] (SEQ ID NOS:1 and 2)

Synonyms: alpha-1 protease inhibitor, alpha-1-antiproteinase

Precursor description: Alpha-1 protease inhibitor (A1AT) is an inhibitor of serine proteases (acc. no. P01009). Its primary target is elastase, but it also has a moderate affinity for plasmin and thrombin. A1AT is a hepatic acute-phase protein, which is required to limit the damage caused by activated neutrophil granulocytes and their enzyme elastase, which breaks down the connective tissue fiber elastin. The expression of A1AT in liver cells is regulated by IL-6 (Morgan et al. 2002). The reactive center loop (RCL) extends out from the body of the protein and directs binding to the target protease. The protease cleaves the serpin at the reactive site within the RCL, establishing a covalent linkage between the carboxyl group of the serpin reactive site and the serine hydroxyl of the protease. The resulting inactive serpin-protease complex is highly stable.

Peptide Description: Two peptides (aa 379-418 =F080.4546.5 (SEQ ID NO:2); aa 383-418=F077.2067.5 (SEQ ID NO:1)) were sequenced during the course of the project. Both peptides contain the reactive center loop (RCL) and represent the C-terminal part of the protein. A1AT undergoes post-translational modifications to yield by-products with novel biological activity. One such molecule, the C-terminal fragment of A1AT, the C-36 peptide (SIPPEVKFNKPFVFLMIEQNTKSPLFMGKWNPTQK (SEQ ID NO:1)) has been reported to stimulate significant pro-inflammatory activity in monocytes and neutrophils in vitro (Bironaite et al. 2001). The peptide corresponds to candidate F080.4546.5.

Polymorphisms: The sequence shown is that of the M1V allele which is the most common form of PI (44 to 49%). Other frequent alleles are: M1A 20 to 23%; M2 10 to 11%; M3 14 to 19%. M1A, a normal variant, is believed to be the ‘oldest’ human PI allele, with the other common normal alleles M1V, M2, and M3 derived from M1A by single base substitutions. M2 is derived from M3; it has the same amino acid difference that distinguishes M3 from M1V but a second substitution in addition. The 4 common normal alleles are considered the ‘base’ from which all the other alleles are derived.

Peptide levels: Both peptides were found to be up-regulated in samples deriving from patients with CLE and who developed a lesion. The peptides were down regulated in healthy individuals.

Biomarker type: Polymorphism, predictive

Disease context: One of the identified peptides corresponds to the C-36 peptide, which has been shown to have atherogenic and inflammatory properties. The peptide may be indicate inflammatory processes in CLE responder patients before lesion development.

TABLE 17
Alignment of the alpha-1-antitrypsin peptides against the
sequence of the precursor

```
A1AT_HUMAN
ENEDRRSASLHLPKLSITGTYDLKSVLGQLGITKVFSNGADLSGVTEEAPLKLSKAVHKA
                        F080.4546.5 -----------------------------------------
-------------------
                        F077.2067.5 -----------------------------------------
------------------- (SEQ ID NO: 2)
                        A1AT_HUMAN
VLTIDEKGTEAAGAMFLEAIPMSIPPEVKFNKPFVFLMIEQNTKSPLFMGKVVNPTQK
                        F080.4546.5 ----------------------
SIPPEVKFNKPFVFLMIEQNTKSPLFMGKVVNPTQK
(SEQ ID NO: 1)
                        F077.2067.5 ------------------
AIPMSIPPEVKFNKPFVFLMIEQNTKSPLFMGKVVNPTQK
(SEQ ID NO: 2)
```

Polymorphism: A deeper investigation of the sequence reveals that the regulation is explainable due to a misbalanced distribution of allelic phenotypes of M1 (400 E) and M2/M3 (400 D). Therefore, each individual was classified according to the phenotype (heterozygote individuals were classified as M2/M3). This normal allele (PI, M1A) has a frequency of 0.44-0.49 in U.S. Caucasians, whereas the M2 and M3 alleles have a frequency of 0.10-0.11 and 0.14-0.19, respectively.

TABLE 18
Classification of individuals according to
the alpha-1-antitrypsin polymorphism

| | 2nd analysis | | 1st analysis | | All | |
|---|---|---|---|---|---|---|
| | Individ- | Lesion | Individ- | Lesion | Individ- | Lesion |
| | uals (n) | (n) | uals (n) | (n) | uals (n) | (n) |
| 400 E | | | | | | |
| HV | 2 | | 1 | | 3 | |
| DLE | 10 | 7 | 8 | 5 | 18 | 12 |
| SCLE | 4 | 1 | 2 | 2 | 6 | 3 |
| LET | 8 | 4 | 2 | 2 | 10 | 6 |
| SUM | 24 | 12 | 13 | 9 | 37 | 21 |
| 400 D | | | | | | |
| HV | 8 | | 2 | | 10 | |
| DLE | 5 | 0 | 2 | 0 | 7 | 0 |
| SCLE | 6 | 4 | 3 | 2 | 9 | 6 |
| LET | 4 | 2 | 1 | 1 | 5 | 3 |
| SUM | 23 | 6 | 8 | 3 | 31 | 9 |

Table 18 above depicts the number of individuals and the number of lesions in each subgroup from both analyses. The subjects with the DLE type, who possess the M2/M3 type did not develop a lesion in both analyses (bold/italic). Since the n-number is very small (n=7) a definitive conclusion cannot be made.

Since both interest list candidates representing peptides with glutamic acid in amino acid position 400 the regulation can be explained due to the distribution of the allelic variants in the different group: In the second analysis 80% of the healthy individuals possess a M2/M3 variant. Therefore, the M1 variant appears to be less frequent in the group of healthy individuals. Furthermore, subjects with a DLE type, who possess the M2/M3 variant did not develop a lesion in both analyses. Therefore, the M1 variant seems to be more frequent in patients who develop a lesion.

### Kininogen-1 [Precursor] (SEQ ID NOS:19-22)

Synonym: Alpha-2-thiol proteinase inhibitor

Precursor description: Human blood plasma contains two kininogens: high-molecular-weight and low-molecular-weight kininogens (HMK and LMK, respectively). Their synthesis is encoded by the same gene located in the third chromosome. Kininogens are polyfunctional glycoproteins consisting of a single polypeptide chain; they are mainly synthesized by hepatocytes and are post-translationally glycosylated and released into the blood flow. The HMK molecule (acc. no. P01042) consists of 626 amino acid residues. Its concentration in human blood plasma is 65-130 μg/ml. The structure of the kininogen molecule is presented in Table 19 below.

The kinins, bradykinin and lysylbradykinin, are important mediators of inflammatory responses. The kinins are potent vasoactive basic peptides and their properties are wide ranging, including the ability to increase vascular permeability, cause vasodilation, pain, and the contraction of smooth muscle, and to stimulate arachidonic acid metabolism.

Peptide description: Three peptides originate from HMK domain 5. Two peptides (F020.1980.5=aa 458-476 (SEQ ID NO:20) and F020.1923.5=aa 459-476 (SEQ ID NO:22)) reside from the histidine-glycine-rich region of the light chain of cleaved HMK. Using synthetic peptides it has been shown that this region is responsible for binding to negatively charged surfaces and initiation of the intrinsic coagulation, fibrinolytic, and kinin-forming systems. The third peptide (aa 381-398=F048.1076.5 (SEQ ID NO:21)) and to the bradykinin molecule and contains a hydroxyproline at position 383. Hydroxylation occurs prior to the release of bradykinin.

Peptide levels: All four identified kininogen peptides are only increased in those CLE patients who do not develop a lesion after photoprovocation (non-responder). The peptides have a similar response pattern as complement C3 peptides.

Biomarker type: Protective

Disease context: When leucocytes migrate from the bloodstream into sites of inflammation or injury, they undergo a complex sequence of adhesion and locomotion steps. These highly coordinated processes require the expression and up-regulation of various adhesion receptors on the surface of leukocytes and vascular cells. Different receptor systems direct the interaction of leukocytes with the endothelium. Firm adhesion to and transmigration through the endothelium are mediated by the β2-integrins Mac-1 (CD11b/CD18, αMβ2, CR3) and LFA-1 (CD11a/CD18, αLβ2), which interact with their counter-receptor ICAM-1 on the endothelial cells. Mac-1 also regulates leukocyte adhesion to provisional matrix substrates, including fibrinogen, which becomes deposited at the sites of inflammation and injury after increases in vascular permeability and damage. Recently, it was shown that peptide fragments from domain 3 and 5 of HMK, but not bradykinin, release the cytokines TNF-α, ilL-1β, IL-6, and the chemokines IL-8 and MCP-1 from isolated human mononuclear cells. However, peptides from domain 5 specifically interact with Mac-1 but not with LFA-1, thereby blocking Mac-1-dependent leukocyte adhesion to fibrinogen and endothelial cells in vitro and in vivo and serving as a novel endogenous regulator of leukocyte recruitment into the inflamed tissue. Here, peptides have been identified from the same region that blocks leukocyte recruitment to inflamed tissues. The peptides are only increased after photoprovocation in CLE patients who do not develop a lesion and may serve as a protective factor to prevent lesion development.

TABLE 20
Alignment of the kininogen peptides against the
sequence of the precursor

```
                        KNG1_HUMAN
EKKIYPTVNCQPLGMISLMKRPPGFSPFRSSRIGEIKEETTVSPPHTSMAPAQDEERDSG
                        F048.1076.5 --------------------RPPGFSPFR-------------
------------------
(SEQ ID NO: 21)
                        F042.6687.5 -----------------------------
SSRIGEIKEETTVSPPHTSMAPAQDEERDSG
(SEQ ID NO: 19)
                        F020.1980.5 ------------------------------------------
------------------
(SEQ ID NO: 20)
                        F020.1923.5 ------------------------------------------
------------------
(SEQ ID NO: 22)
                        KNG1_HUMAN
KEQGHTRRHDWGHEKQRKHNLGHGHKHERDQGHGHQRGHGLGHGHEQQHGLGHGHKFKLD
                        F048.1076.5 ------------------------------------------
------------------
(SEQ ID NO: 21)
                        F042.6687.5 KEQGHTRRHDWGHEKQ--------------------------
------------------
(SEQ ID NO: 19)
                        F020.1980.5 -------------------------------------
GHGLGHGHEQQHGLGHGHK----
(SEQ ID NO: 20)
                        F020.1923.5 --------------------------------------
HGLGHGHEQQHGLGHGHK----
(SEQ ID NO: 22)
The peptide at aa 381-389 (SEQ ID NO: 21) (highlighted in bold) comprise bradykinin. The kininogen-1 light chain is underlined.
```

### Collagens

In total, ten peptides from collagen precursors were identified: five peptides from collagen type I, one from collagen type II, three from collagen type III, one from collagen type IV and one from collagen type V. Table 14 lists the tissue expression of the identified collagens. All peptides are fragments of the triple-helical region of the collagen isotypes having high proline content and with frequently hydroxylated proline residues.

Disease context: With the exception of one peptide from the collagen type V, most other collagen peptides were decreased in CLE responders either before lesion development and/or during disease flares. Peptides from the triple-helical region may be viewed as biochemical markers for collagen turnover and/or production. It has been shown that INF-_{y} suppresses collagen synthesis in several tissues suggesting that CLE responders to photoprovocation may have increased levels of INF-g before photoprovocation.

It was also reported that SLE patients have a higher risk for osteoporosis and lower bone mineral density, which can be related to corticosteroid therapy. On the other hand it was reported that IL-6 is a predictor for bone loss in post-menopausal women and that IL-6 treatment of osteoclast induces bone resorption suggesting also a role for IL-6 in collagen metabolism.

TABLE 21
Tissue expression and function of identified collagen precursors

| Type | Notes | Gene(s) |
|---|---|---|
| I | 90% of the collagen in | COL1A1, COL1A2 |
| | human body; present in | |
| | bone, skin (associated with | |
| | type III collagen6) and | |
| | tendons | |
| III | the major collagen found in | COL3A1 |
| | skin, blood vessels and | |
| | internal organs such as the | |
| | smooth muscle layers of the | |
| | gastrointestinal tract | |
| IV | basal lamina; eye lens | COL4A1, COL4A2, |
| | | COL4A3, COL4A4, |
| | | COL4A5, COL4A6 |
| V | a minor collagen as it is | COL5A1, COL5A2, |
| | present in less than 10% of | COL5A3 |
| | the total collagen in any | |
| | tissue; usually found with | |
| | type I collagen and type III | |
| | collagen in bone, tendon, | |
| | cornea, skin, blood vessels | |
| | and lungs; during foetal | |
| | development, basement | |
| | membranes originally | |
| | contain type V collagen | |
| | before being replaced to | |
| | type IV collagen | |

### Collagen Alpha-1(1) Chain [Precursor]

Precursor description: Type I collagen (acc. no. P02452) is a member of group I collagen (fibrillar forming collagen).

Peptide description: Four peptide fragments (F042.1611.5=aa 400-417 (SEQ ID NO:26)), F043.2481.5=aa 478-504 (SEQ ID NO:27), F026.1938.5=aa 979-999 (SEQ ID NO:28), F029.2940.5=aa 220-249)(SEQ ID NO:29)) from the triple helical region of CO1A1 (aa 179-1192) were identified.

Peptide levels: The level of all four peptides is decreased in CLE patients developing a lesion.

Biomarker type: Predictive

### Collagen Alpha-2(1) Chain [Precursor]

Precursor description: The collagen alpha-2(I) chain (acc. no. P08123, 1366 amino acids, molecular mass 129,412) is a member of group I collagen (fibrillar forming collagen) and forms trimers of one alpha 2(1) and two alpha 1(I) chains. It forms the fibrils of tendon, ligaments and bones. In bones the fibrils are mineralized with calcium hydroxyapatite.

Peptide description: The identified peptide (F026.2823.5) is a fragment of the Collagen alpha-2(I) chain (aa 80-1102) (SEQ ID NO:8).

Peptide levels: The level of the peptide is lower before photoprovocation in CLE patients who develop a lesion (responders).

Biomarker type: Predictive

### Collagen Alpha-1(III) Chain [Precursor] (SEQ ID NOS:30-32)

Precursor description: Collagen type III belongs to the fibrillar collagens and occurs in most soft connective tissues along with type I collagen. (acc. no. P02461)

Peptide description: All three identified peptides (F029.2353.5=aa 187-211 (SEQ ID NO:30), F033.3025.5=aa 319-351 (SEQ ID NO:31), F034.2825.5=aa 694-726 (SEQ ID NO:32)) originate from the triple-helical region (aa 149-1205).

Peptide levels: All identified peptides are decreased in CLE patients who develop lesions before or/and after photoprovocation. Two peptides showed an increase in non-responders after photoprovocation.

Biomarker type: Predictive

### Collagen Alpha-1(IV) Chain [Precursor] (SEQ ID NO:46)

Precursor description: Type IV collagen is the major structural component of glomerular basement membranes (GBM), forming a ‘chicken-wire’ meshwork together with laminins, proteoglycans and entactin/nidogen. There are six type IV collagen isoforms, alpha 1(IV)-alpha 6(IV), each of which can form a triple helix structure with 2 other chains to generate type IV collagen network. (acc. no. P02462).

Peptide description: The identified peptide (F021.1192.5=aa 410-422)(SEQ ID NO:46) originates from the triple-helical region (aa 173-1440).

Peptide levels: The peptide is increased after UV-radiation in CLE patients who do not develop a lesion (non-responder).

Biomarker type: Lesion

### Collagen Alpha-1(V) Chain [Precursor] (SEQ ID NO:33)

Precursor description: Type V collagen (acc. no. P20908, 1,838 amino acids, molecular mass 183,560) is a member of group I collagen (fibrillar forming collagen). It is a minor connective tissue component of nearly ubiquitous distribution. Type V collagen binds to DNA, heparan sulfate, thrombospondin, heparin, and insulin.

Peptide description: The peptide (F026.2443.5=aa 457-481) is a fragment of the junction between the interrupted collagenous region (aa 444-558) and the triple-helical region (aa 559-1570).

Peptide levels: Levels of the peptide are increased in both CLE non-responders and responders to photoprovocation as compared to healthy subjects.

### Miscellaneous

### Sulfhydryl Oxidase 1 [Precursor] (SEQ ID NO:38)

Synonyms:EC 1.8.3.2, Quiescin Q6, hQSOX

Precursor description: ‘Quiescin Q6’ describes that it was the sixth clone to be found at a higher level of expression in quiescent fibroblasts (acc. no. 000391). This protein is expressed in heart, placenta, lung, liver, skeletal muscle, pancreas and very weakly in brain and kidney. It catalyzes the oxidation of sulfhydryl groups in peptide and protein thiols to disulfides with the reduction of oxygen to hydrogen peroxide. It may contribute to disulfide bond formation in a variety of secreted proteins. In fibroblasts, it may have tumor-suppressing capabilities being involved in growth regulation. Two isoforms have been identified: Isoform 1 is predicted to be a membrane single-pass membrane protein, whereas isoform 2 is a secreted protein.

Peptide description: The identified peptide (F055.3324.5) covers a region, which is only present in isoform 1.

Peptide levels: The level of the peptide is decreased in patients who develop a lesion after photoprovocation (responder).

Biomarker type: Lesion

TABLE 22
Alignment of the Quiescin Q6 peptide against the
sequence of the precursor

```
                QSCN6_HUMAN
PEASRPPKLHPGLRAAPGQEPPEHMAELQRNEQEQPLGQWHLSKRDTGAALLAESRAEKN
                F055.3324.5 --------------
AAPGQEPPEHMAELQRNEQEQPLGQWHLS-----------------
(SEQ ID NO: 38)
                QSCN6_HUMAN
RLWGPLEVRRVGRSSKQLVDIPEGQLEARAGRGRGQWLQVLGGGFSYLDISLCVGLYSLS
                F055.3324.5 -----------------------------------------
-------------------
(SEQ ID NO: 38)
                QSCN6_HUMAN FMGLLAMYTYFQAKIRALKGHAGHPAA
                F055.3324.5 ---------------------------
Underlined amino acids are missing in isoform 2, bold potential transmembrane site
```

### Albumin [Precursor] (SEQ ID NOS:36 and 37)

Precursor description: Serum albumin (acc. no. P02768), the main protein of plasma, has a high binding capacity for water, Ca²⁺, Na⁺4 K⁺, fatty acids, hormones, bilirubin and drugs. Its main function is the regulation of the colloidal osmotic pressure of blood. Serum albumin has potent antioxidant properties, probably due to binding of copper and other transition metals. In normal (intact) human albumin, the N-terminal region comprised of the amino-acid sequence N-Asp-Ala-His-Lys has been shown to be a strong binding site for transition metals such as Co²⁺, Cu²⁺ and Ni²⁺.

Peptide description: One peptide F043.2357.5 (aa 25-44) (SEQ ID NO:37) originates from the N-terminus of human serum albumin. It covers the Copper binding His27 and tetrapeptide DAHK has been shown to provide neuroprotection by limiting limiting metal-catalyzed oxidant stress.

Peptide levels: Peptide F040.1434.5 (551-563) (SEQ ID NO:36) is increased in CLE patients developing a lesion, whereas the peptide F043.2357.5 (aa 25-44) is downregulated in CLE patients developing a lesion. Thus, it cannot be concluded that the levels of the albumin precursor protein are altered in CLE patients developing a lesion. The peptides may rather reflect differential degradation pathways associated with the onset of lesions.

Biomarker type: Predictive

TABLE 23
Alignment of the albumin peptides against the
sequence of the precursor

ALBU_HUMAN
MKWVTFISLLFLFSSAYSRGVFRRDAHKSEVAHRFKDLGEENFKALVLIAFAQYLQQCPF
F040.1434.5 ------------------------------------------------------
------
F043.2357.5 ------------------------DAHKSEVAHRFKDLGEENFK----------
------(SEQ ID NO: 37)
-----------------------------//------------------------
------
ALBU_HUMAN
SEKERQIKKQTALVELVKHKPKATKEQLKAVMDDFAAFVEKCCKADDKETCFAEEGKKLV
F040.1434.5 ----------TALVELVKHKPKA-------------------------------
------(SEQ ID NO: 36)
F043.2357.5 ------------------------------------------------------
------
The propeptide is highlighted in bold, the copper-binding site in italics.

### Apolipoprotein C-I [Precursor] (SEQ ID NO:4)

Precursor description: Apolipoprotein (apo)C-I (acc. no. P02654) is synthesized mainly in the liver and to a minor degree in the intestine. It is a constituent of high density lipoproteins (HDL) and of triglyceride-rich lipoproteins that slow the clearance of triglyceride-rich lipoproteins by a variety of mechanisms. ApoC-I is an inhibitor of lipoprotein binding to the LDL receptor, LDL receptor-related protein, and VLDL receptor. It also is the major plasma inhibitor of cholesteryl ester transfer protein, and appears to interfere directly with fatty acid uptake.

Peptide description: The peptide (F055.1505.5=aa 29-42) (SEQ ID NO:4) is a fragment of the mature ApoC-I chain.

Peptide levels: The level of the peptide is higher in CLE patients who do not develop lesions (non-responder).

Biomarker type: Protective

Disease context: The host response in inflammation is accompanied by profound alterations in lipid metabolism and hence the distribution and composition of lipoprotein subclasses. Especially HDL, which mainly consist of apolipoproteins and phospholipids and that represent the most frequent lipoproteins in human plasma, have been demonstrated to play an important physiological role in restricting the harmful effects of inflammation and infection. HDL has not only been shown to mediate reverse cholesterol transport but also the clearance of inflammatory mediators such as bacterial lipopolysaccharide or the scavenging of oxidation products, thereby contributing to tissue integrity. In postsurgery patients with systemic inflammatory response or sepsis, an almost total loss of apoC-I was described. Thus, alterations of HDL concentration and subclass composition, may be detrimental in systemic inflammation.

TABLE 24
Alignment of the ApoC-I peptide against the
sequence of the precursor

APOC1_HUMAN
MRLFLSLPVLVVVLSIVLEGPAPAQGTPDVSSALDKLKEFGNTLEDKARELISRIKQSEL
F055.1505.5 ----------------------------DVSSALDKLKEFGN------------
------
(SEQ ID NO: 4)
APOC1_HUMAN SAKMREWFSETFQKVKEKLKIDS
F055.1505.5 -----------------------
(SEQ ID NO: 4)
The sequence of the mature ApoC-I molecule (aa 27-83) is highlighted in bold.

### Hemoglobin Alpha Chain [Precursor] (SEQ ID NO:35)

Precursor description: Hemoblobin is responsible for the transport of oxygen from the lung to the various peripheral tissues. It constitutes a heterotetramer of two alpha chains and two beta chains in adult hemoglobin A (HbA); two alpha chains and two delta chains in adult hemoglobin A2 (HbA2). The hemoglobin alpha chain (acc. no. P69905)

Peptide description: One peptide (F054.1699.5) was identified from the hemoglobin alpha chain precursor covering aa 127-141.

Peptide levels: The level of the peptide is lower in CLE patient who do not develop a lesion (non-responders) compared to controls and CLE patients who develop a lesion (responders).

Biomarker type: Predictive

Result interpretation: The appearance of the peptide may be associated with changes in hemoglobin levels, hemolysis during blood sampling or reduced stabilities of erythrocytes. It has been shown that recombinant IL-6 can induce anemia.

TABLE 25
Alignment of the hemoglobin peptide against the sequence of the precursor

| | |
|---|---|
| 1 | VLSPADKTNV KAAWGKVGAH AGEYGAEALE |
| | RMFLSFPTTK TYFPHFDLSH GSAQVKGHGK |
| 61 | KVADALTNAV AHVDDMPNAL SALSDLHAHK |
| | LRVDPVNFKL LSHCLLVTLA AHLPAEFTPA |
| 121 | VHASLD KFLA SVSTVLTSKY R |

The identified peptide is highlighted in bold.

TABLE 26
Interpretation of the findings in the context of CLE or SLE

| Precursor | Type of | Regu- | | | | |
|---|---|---|---|---|---|---|
| Name | biomarker | lation | IL-6 | Cytokines | CLE | SLE |
| Alpha-1- | Poly- | ↑ R | increased | | C-36 peptide stimulates | C-36 peptide stimulates |
| antitrypsin | morphism/ | | | | monocyte cytokine | monocyte cytokine |
| | Predictive | | | | release (TNF-α, IL-1b, | release (TNF-α, IL-1b, |
| | | | | | IL-8), E800 allel is | IL-8) |
| | | | | | more frequent in | |
| | | | | | responders | |
| Alpha-2- | Predictive* | ↑ R | increased | | abnormalities in | abnormalities in |
| antiplasmin | | | | | coagulation and | coagulation and |
| | | | | | fbrinolysis | fbrinolysis |
| Fibrinogen | Predictive | ↑ R | increased | | abnormalities in | abnormalities in |
| alpha chain | | | | | coagulation and | coagulation and |
| | | | | | fbrinolysis | fbrinolysis |
| Fibrinogen | Lesion | ↓ R | increased | | abnormalities in | abnormalities in |
| beta chain | | | | | coagulation and | coagulation and |
| | | | | | fbrinolysis | fbrinolysis |
| Hemoglobin | Predictive | ↑ R | indirect | | | IL-6 correlates with |
| alpha chain | | | | | | anemia, decrease in |
| | | | | | | iron supply |
| Complement | Lesion | ↑ R post | increased | | Disease activity | Disease activity |
| C3 | | | | | marker, cleavage of | marker, cleavage of |
| | | | | | C3b produces C3f and | C3b produces C3f and |
| | | | | | C3bi, which is | C3bi, which is |
| | | | | | important for | important for |
| | | | | | maintaining B-cell | maintaining B-cell |
| | | | | | tolerance | tolerance |
| Serum | Predictive | ↓ R | decreased | | | |
| albumin | | | | | | |
| Collagen | Predictive | ↓ R | indirect | inflammatory | decreased osteoblast | osteoporosis and |
| | | | | cytokines | and increased | osteonecrosis |
| | | | | (INF-γ, TNF-α) | osteoclast number and | |
| | | | | decrease collagen | activity | |
| | | | | synthesis | | |
| Complement | Protective | ↑ NR | increased/ | INF-γ | C4 is protective for | C4 is protective for |
| C4-A | | post | or no | | lupus, maintaining | lupus, maintaining |
| | | | effect | | tolerance to self-Ag, | tolerance to self-Ag, |
| | | | | | uptake of apoptotic | uptake of apoptotic |
| | | | | | cells | cells |
| Kininogen-1 | Protective | ↑ NR | increased | | synthetic peptides | synthetic peptides |
| | | post | | | decrease leukocyte | decrease leukocyte |
| | | | | | recruitment into the | recruitment into the |
| | | | | | imflamed tissue, | imflamed tissue, |
| | | | | | stimulates release of | stimulates release of |
| | | | | | TNF-α, IL-12 | TNF-α, IL-12 |
| Apolipo- | Protective | ↑ NR | no data | | | |
| protein C-I | | post | | | | |
| Sulfhydryl | Lesion* | ↓ R post | unknown | | play a role in growth | play a role in growth |
| oxidase 1 | | | | | arrest | arrest |

While having described the invention in general terms, the embodiments of the invention will be further disclosed in the following examples which should not be construed as limiting the scope of the claims.

TABLE 27

| | SEQ ID | | | | | | | Peptide |
|---|---|---|---|---|---|---|---|---|
| Peptide ID | No | Pre.H | Post.H | Pre.N.R | Post.N.R | Pre.R | Post.R | Type |
| F080.4546.5 Alpha-1-antitrypsin | 1 | 19.35 | 21.33 | 27.78 | 26.95 | 32.57 | 27.48 | Predictive |
| (Polymorphism) | | | | | | | | |
| F077.2067.5 Alpha-1-antitrypsin | 2 | 14.04 | 8.79 | 15.56 | 18.05 | 22.32 | 17.46 | Predictive |
| (Polymorphism) | | | | | | | | |
| F038.1154.5 Alpha-2-antiplasmin | 3 | 338.24 | 356.99 | 316.41 | 300.59 | 416.58 | 421.47 | Predictive |
| F055.1505.5 ApolipoproteinC-I | 4 | 22.04 | 18.77 | 20.46 | 24.67 | 18.46 | 15.88 | |
| F079.11730.5 Beta-2-microglobulin | 5 | 252.19 | 264.67 | 330.00 | 240.90 | 377.28 | 302.48 | Predictive |
| F054.1964.5 Beta-defensin1 | 6 | 73.69 | 65.37 | 56.42 | 53.95 | 86.57 | 64.20 | Predictive |
| F062.3560.5 CD99antigen | 7 | 31.77 | 24.07 | 26.44 | 20.03 | 32.25 | 29.36 | Predictive |
| F021.1192.5 Collagenalpha-1(IV)chain | 8 | 78.16 | 99.32 | 101.24 | 182.60 | 69.67 | 53.28 | |
| F047.1968.5 ComplementC3 | 9 | 32.34 | 22.15 | 25.17 | 23.18 | 27.57 | 30.61 | Protective |
| F036.1499.5 ComplementC4-A | 10 | 1240.79 | 2346.56 | 2172.91 | 2869.71 | 1931.58 | 1900.33 | Protective |
| F046.1896.5 ComplementC4-Aprecursor | 11 | 40.98 | 48.11 | 44.27 | 72.15 | 47.68 | 42.69 | Protective |
| F042.1879.5 ComplementC4-A | 12 | 210.97 | 302.72 | 318.65 | 612.74 | 327.95 | 314.22 | Protective |
| F033.1362.5 ComplementC4-Aprecursor | 13 | 104.75 | 126.21 | 140.85 | 232.29 | 99.89 | 106.62 | Protective |
| F033.1449.5 ComplementC4-A | 14 | 571.17 | 833.58 | 896.45 | 1319.47 | 606.54 | 644.29 | Protective |
| F024.2659.5 Fibrinogenalphachain | 15 | 98.73 | 75.25 | 111.15 | 111.54 | 132.73 | 116.37 | Predictive |
| F039.2769.5 Fibrinogenalphachain | 16 | 92.56 | 85.79 | 136.60 | 115.44 | 179.14 | 162.61 | Predictive |
| F051.4591.5 Fibrinogenbetachain | 17 | 30.02 | 17.65 | 32.83 | 38.73 | 15.60 | 14.90 | Predictive |
| F079.11774.5 IgkappachainV-III | 18 | 120.31 | 148.95 | 152.38 | 111.60 | 184.05 | 154.59 | Predictive |
| F042.6687.5 Kininogen-1 | 19 | 18.34 | 18.47 | 15.39 | 22.63 | 16.22 | 14.71 | Protective |
| F020.1980.5 Kininogen-1 | 20 | 5.89 | 8.56 | 23.65 | 96.65 | 9.53 | 8.70 | Protective |
| F048.1076.5 Kininogen-1 | 21 | 146.32 | 178.55 | 218.43 | 316.77 | 116.00 | 111.31 | Protective |
| F020.1923.5 Kininogen-1 | 22 | 4.39 | 6.68 | 14.33 | 61.02 | 5.33 | 4.31 | Protective |
| F055.3834.5 Polymeric-immunoglobulinreceptor | 23 | 51.66 | 38.35 | 43.72 | 41.61 | 54.82 | 52.89 | Predictive |
| F060.5233.5 Polymeric-immunoglobulinreceptor | 24 | 11.58 | 8.93 | 13.16 | 8.08 | 14.23 | 17.34 | Predictive |
| F046.4352.5 Polymeric-immunoglobulinreceptor | 25 | 19.91 | 16.17 | 16.80 | 15.95 | 26.40 | 23.78 | Predictive |
| F042.1611.5 Collagen alpha-1(I) chain | 26 | 16.75 | 15.50 | 11.33 | 15.25 | 10.74 | 11.30 | Predictive |
| F043.2481.5 Collagen alpha-1(I) chain | 27 | 74.94 | 71.37 | 67.40 | 73.73 | 54.48 | 58.18 | Predictive |
| F026.1938.5 Collagen alpha-1(I) chain | 28 | 48.84 | 56.11 | 62.34 | 54.91 | 46.47 | 57.28 | Predictive |
| F029.2940.5 Collagen alpha-1(I) chain | 29 | 35.85 | 39.70 | 34.21 | 35.40 | 26.65 | 29.44 | Predictive |
| F029.2353.5 Collagen alpha-1(III) chain | 30 | 76.38 | 77.01 | 86.49 | 72.84 | 63.05 | 83.77 | Predictive |
| F033.3025.5 Collagen alpha-1(III) chain | 31 | 18.14 | 18.73 | 18.52 | 18.85 | 16.83 | 13.15 | Predictive |
| F034.2825.5 Collagen alpha-1(III) chain | 32 | 16.58 | 16.04 | 18.60 | 21.26 | 16.56 | 13.73 | |
| F026.2443.5 Collagen alpha-1(V) chain | 33 | 29.12 | 30.19 | 39.89 | 31.60 | 37.73 | 40.76 | Predictive |
| F026.2823.5 Collagen alpha-2(I) chain | 34 | 35.90 | 39.82 | 39.72 | 35.87 | 29.68 | 38.09 | Predictive |
| F054.1699.5 Hemoglobin alpha chain | 35 | 27.05 | 16.34 | 12.68 | 12.14 | 26.25 | 20.14 | |
| F040.1434.5 Serum albumin | 36 | 30.62 | 33.99 | 36.96 | 36.77 | 42.65 | 43.52 | |
| F043.2357.5 Serum albumin | 37 | 41.27 | 54.47 | 48.64 | 38.72 | 26.87 | 40.96 | |
| F055.3324.5 Sulfhydryl oxidase 1 | 38 | 45.34 | 46.02 | 41.06 | 50.79 | 42.79 | 37.24 | |

Pre.H and Post.h are healthy subjects
Pre.N.R., Post.N.R., Pre.R and Post.R are CLE Subjects

Although illustrated and described above with reference to certain specific embodiments, the present invention is nevertheless not intended to be limited to the details shown. Rather, the present invention is directed to the lupus-related genes and gene products. Polynucleotides, antibodies, apparatus, and kits disclosed herein and uses thereof, and methods for predicting responsiveness to treatment and controlling the levels of the lupus-related biomarker genes, and various modifications may be made in the details within the scope and range of equivalents of the claims and without departing from the spirit of the invention.

What is claimed:

1. A method for detecting susceptibility to lupus or CLE in a subject, comprising the steps of:
isolating a first biological sample from a subject;
exposing the subject to photoprovocation;
isolating a second biological sample from the subject exposed to photoprovocation;
comparing level(s) of at least one lupus-related photoprovocation marker peptide selected from the group consisting of all or a portion of the amino acid sequences set forth in SEQ ID NOS: 1-7, 15-18, 23-31, 33 and 34 from the first biological sample to level(s) from the second biological sample; and
identifying an altered concentration of at least one lupus-related photoprovocation marker peptide, wherein the altered concentration showing a statistically significant increase in the at least one lupus-related photoprovocation marker peptide indicates susceptibility to lupus.

2. The method of claim 1, wherein the sample comprises plasma or serum from the subject.

3. The method of claim 1, wherein the sample comprises peripheral blood cells.

4. The method of claim 1, wherein the at least one lupus-related photoprovocation marker peptide is isolated from plasma of the subject and is a predictive biomarker.

5. The method of claim 1, wherein the at least one marker peptide is an array of nucleic acid segments.

6. The method of claim 5, wherein the testing is done by RT-PCR.

7. The method of claim 5, wherein the testing is done by ELISA.

8. A kit for prognostic or diagnostic use, comprising an oligonucleotide comprising at least 15 nucleotides comprising or complementary to a polynucleotide comprising the nucleotide sequence of a marker gene or the complementary strand thereof and cells expressing the marker gene, wherein the marker gene is selected from the group consisting of all or a portion of the nucleotide sequences encoding the amino acid sequences set forth in SEQ ID NOS: 1-7, 15-18, 23-31, 33 and 34.

9. A kit for screening a subject for susceptibility to lupus or lupus-related skin lesions, the kit comprising an antibody which recognizes a predictor peptide comprising an amino acid sequence selected from the group consisting of all or a portion of SEQ ID NOS: 1-7, 15-18, 23-31, 33 and 34.

10. The kit of claim 9, wherein the predictor peptide is encoded by a marker gene and cells expressing the marker gene, wherein the marker gene is selected from the group consisting of all or a portion of the nucleotide sequences encoding the amino acid sequences set forth in SEQ ID NOS: 1-7, 15-18, 23-31, 33 and 34.

11. A method for detecting susceptibility to lupus or CLE in a subject, comprising the steps of:
isolating a first biological sample from a subject;
exposing the subject to photoprovocation;
isolating a second biological sample from the subject exposed to photoprovocation;
comparing level(s) of at least one lupus-related photoprovocation marker peptide selected from the group consisting of all or a portion of the amino acid sequences set forth in SEQ ID NOS:9-14 and 19-22 from the first biological sample to level(s) from the second biological sample; and
identifying an altered concentration of at least one lupus-related photoprovocation marker peptide, wherein the altered concentration being an increase in the at least one lupus-related photoprovocation marker peptide indicates a lack of susceptibility to lupus.

12. The method of claim 11, wherein the sample comprises plasma or serum from the subject.

13. The method of claim 11, wherein the sample comprises peripheral blood cells.

14. The method of claim 11, wherein the at least one lupus-related photoprovocation marker peptide is isolated from plasma of the subject and is a protective biomarker.

15. The method of claim 11, wherein the at least one marker peptide is an array of nucleic acid segments.

16. The method of claim 15, wherein the testing is done by RT-PCR.

17. The method of claim 15, wherein the testing is done by ELISA.

18. A kit for prognostic or diagnostic use, comprising an oligonucleotide comprising at least 15 nucleotides comprising or complementary to a polynucleotide comprising the nucleotide sequence of a marker gene or the complementary strand thereof and cells expressing the marker gene, wherein the marker gene is selected from the group consisting of all or a portion of the nucleotide sequences encoding the amino acid sequences set forth in SEQ ID NOS: 9-14 and 19-22.

19. A kit for screening a subject for susceptibility to lupus or lupus-related skin lesions, the kit comprising an antibody which recognizes a predictor peptide comprising an amino acid sequence set forth in SEQ ID NOS: 9-14 and 19-22.

20. The kit of claim 19, wherein the predictor peptide is encoded by a marker gene and cells expressing the marker gene, wherein the marker gene is selected from the group consisting of all or a portion of the nucleotide sequences encoding the amino acid sequences set forth in SEQ ID NOS: 9-14 and 19-22.

21. A method for detecting susceptibility to lupus-related skin lesions in a lupus patient, comprising the steps of:
isolating a first biological sample from a lupus patient;
exposing the patient to photoprovocation;
isolating a second biological sample from the patient exposed to photoprovocation;
comparing level(s) of at least one lupus-related photoprovocation marker peptide selected from the group consisting of all or a portion of the amino acid sequences set forth in SEQ ID NOS: 1-44 from the first biological sample to level(s) from the second biological sample; and
identifying an altered concentration of at least one lupus-related photoprovocation marker peptide, wherein the altered concentration being an increase or decrease in the at least one lupus-related photoprovocation marker peptide indicates susceptibility to lupus-related skin lesions.

22. The method of claim 21, wherein the at least one lupus-related photoprovocation marker peptide is selected from the group consisting of all or a portion of SEQ ID NOS: 1-7, 15-18, 23-31, 33 and 34.

23. The method of claim 21, wherein the at least one lupus-related photoprovocation marker peptide is selected from the group consisting of all or a portion of SEQ ID NOS: 9-14 and 19-22

24. A method for detecting susceptibility to lupus, CLE, or lupus-related skin lesions in a subject, comprising the steps of:
isolating a biological sample from the subject;
comparing level(s) of at least one lupus-related photoprovocation marker peptide selected from the group consisting of all or a portion of the amino acid sequences set forth in SEQ ID NOS: 9-14 and 19-22 from the biological sample to level(s) from a control group; and
identifying an altered concentration of the at least one lupus-related photoprovocation marker peptide compared to the control group level, wherein the altered concentration being an increase in the at least one lupus-related photoprovocation marker peptide indicates susceptibility to lupus, CLE, or lupus-related skin lesions.

25. The method of claim 24, wherein the sample comprises plasma or serum from the subject.

26. The method of claim 24, wherein the sample comprises peripheral blood cells.

27. The method of claim 24, wherein the at least one lupus-related photoprovocation marker peptide is isolated from plasma of the subject and is a predictive biomarker.

28. The method of claim 24, wherein the at least one marker peptide is an array of nucleic acid segments.

29. The method of claim 28, wherein the testing is done by RT-PCR.

30. The method of claim 28, wherein the testing is done by ELISA.

31. The method of claim 24, when the marker peptide is compared to a control, said elevated concentration of said at least one marker peptide being indicative of the existence of susceptibility to developing lupus or lupus-related skin lesions in said subject or patient.

32. The method of claim 24, wherein the step of identifying comprises the step of determining a relative concentration of said at least one marker peptide, compared with a concentration of the same peptide in a control sample, wherein a concentration change with an error probability of less than at least 90% is regarded as a positive detection result for the existence of susceptibility to developing lupus or lupus-related skin lesions.

33. The method of claim 24, wherein the at least one lupus-related photoprovocation marker peptide is in unmodified form, in chemically modified form or has post-translational modifications selected from the group consisting of phosphorylation and addition of an N-terminal pryoglutamic acid group.

34. The method of claim 24, wherein the biological sample is from plasma.

35. The method of claim 24, wherein the step of identifying is carried out with the aid of a mass spectrometric determination.

36. The method of claim 35, wherein the mass spectrometric determination is made with a MALDI (matrix-assisted laser desorption and ionization) mass spectrometry.

37. The method of claim 24, wherein the at least one lupus-related photoprovocation marker peptide is identified with the aid of an immunological, physical or chemical test.

38. The method of claim 24, wherein the at least one marker peptide comprises an array of nucleic acid segments.

39. The method of claim 24, wherein the comparing step comprises evaluating the sample against a reference standard and determining whether the average intensity value for the at least one marker peptide is equal to or above X or below Y.

40. The method of claim 39, wherein the average intensity value for the at least one marker peptidel being equal to or above X indicates the subject will be a responder to the target therapy and the average intensity value for the at least one marker peptide being below Y indicates the subject will be a non-responder.

41. The method of claim 40, further comprising after the identifying step, treating the subject with a therapy based on the average intensity value for the at least one marker peptide being equal to or above X.

42. The method of claim 40, further comprising after the identifying step, refraining from treating the subject with a therapy based on the average intensity value for the at least one marker peptide being less than Y.

43. A method for predicting whether a lupus or CLE patient will respond to treatment with a therapeutic agent, comprising isolating a first biological sample from the patient;
exposing the patient to photoprovocation;
isolating a second biological sample from the patient exposed to photoprovocation;
comparing level(s) of at least one lupus-related photoprovocation marker peptide selected from the group consisting of all or a portion of the amino acid sequences set forth in SEQ ID NOS:1-45 from the first biological sample to level(s) from the second biological sample; and
identifying an altered concentration of the at least one lupus-related photoprovocation marker peptide, wherein the altered concentration being statistically significant increase or decrease in the at least one lupus-related photoprovocation marker peptide indicates responsiveness or non-responsiveness to treatment.

44. The method of claim 43, wherein the at least one lupus-related photoprovocation marker peptide is selected from the group consisting of all or a portion of SEQ ID NOS: 1-7, 15-18, 23-31, 33 and 34 and the at least one marker peptide is a predictive biomarker isolated from plasma.

45. The method of claim 43, wherein the at least one lupus-related photoprovocation marker peptide is selected from the group consisting of all or a portion of SEQ ID NOS: 9-14 and 19-22 and the at least one marker peptide is a protective biomarker isolated from plasma.

46. The method of claim 43, wherein the biological sample is cerebrospinal fluid, serum, plasma, skin, urine, synovial fluid, sputum, stool, tear fluid or a tissue homogenate.

47. The method of claim 46, wherein the biological sample is plasma or skin.

48. The method of claim 43, wherein the step of identifying is performed using a mass spectrometric determination.

49. The method of claim 48, wherein the mass spectrometric determination is from MALDI (matrix-assisted laser desorption and ionization) mass spectrometry.

50. The method of claim 43, wherein prior to the step isolating a second biological sample the patient is treated with the therapeutic agent.

51. The method of claim 50, wherein the altered concentration is a statistically significant increase in the at least one lupus-related photoprovocation marker peptide indicating responsiveness to treatment with the therapeutic agent.

52. The method of claim 51, wherein treatment with the therapeutic agent is continued.

53. The method of claim 50, wherein the altered concentration is a statistically significant decrease in the at least one lupus-related photoprovocation marker peptide indicating non-responsiveness to treatment with the therapeutic agent.

54. The method of claim 53, wherein treatment with the therapeutic agent is discontinued.

55. The method of claim 43, wherein the at least one lupus-related photoprovocation marker peptide is identified with the aid of an immunological, physical or chemical test.

56. A method for predicting whether a lupus or CLE patient will respond to treatment with a therapeutic agent, comprising
isolating a biological sample from the patient;
comparing level(s) of at least one lupus-related photoprovocation marker peptide selected from the group consisting of all or a portion of the amino acid sequences set forth in SEQ ID NOS: 1-44 from the biological sample to level(s) from a control group; and
identifying an altered concentration of the at least one lupus-related photoprovocation marker peptide compared to the control group level, wherein the altered concentration being an increase or decrease in the at least one lupus-related photoprovocation marker.

57. The method of claim 56, wherein an increase in the at least one marker peptide indicates responsiveness to treatment.

58. The method of claim 56, wherein a decrease in the at least one marker peptide indicates non-responsiveness to treatment.

59. The method of claim 56, wherein the at least one lupus-related photoprovocation marker is selected from the group consisting of all or a portion of SEQ ID NOS: 1-7, 15-18, 23-31, 33 and 34

60. The method of claim 59, wherein the at least one lupus-related photoprovocation marker is a predictive biomarker from plasma.

61. The method of claim 56, wherein the at least one lupus-related photoprovocation marker comprises an array of nucleic acid segments.

62. The method of claim 56, wherein the comparing step comprises evaluating the sample against a reference standard and determining whether the average intensity value for each of the members of the panel is equal to or above X or below Y.

63. The method of claim 62, wherein the average intensity value for each of the members of the panel being equal to or above X indicates the subject will be a responder to the target therapy and the average intensity value for each of the members of the panel being below Y indicates the subject will be a non-responder.

64. The method of claim 63, further comprising after the identifying step, treating the subject with the target therapy based on the average intensity value for each of the members of the panel being equal to or above X.

65. The method of claim 63, further comprising after the identifying step, refraining from treating the subject with the target therapy based on the average intensity value for each of the members of the panel being less than Y.

66. The method of claim 56, wherein the testing is done by RT-PCR.

67. The method of claim 56, wherein the testing is done by ELISA.

68. A kit for prognostic or diagnostic use, comprising an oligonucleotide comprising at least 15 nucleotides comprising or complementary to a polynucleotide comprising the nucleotide sequence of a marker gene or the complementary strand thereof and cells expressing the marker gene, wherein the marker gene is one or more polynucleotides selected from the group consisting of all or a portion of the nucleotide sequences encoding the amino acid sequences set forth in SEQ ID NOS: 1-45.

69. Any invention described herein.


SEQUENCE LISTING (46 sequences)

SEQ ID NO: 1 - protein, 36 aa, Homo sapiens
SIPPEVKFNKPFVFLMIEQNTKSPLFMGKVVNPTQK

SEQ ID NO: 2 - protein, 40 aa, Homo sapiens
AIPMSIPPEVKFNKPFVFLMIEQNTKSPLFMGKVVNPTQK

SEQ ID NO: 3 - protein, 12 aa, Homo sapiens
MEPLGRQLTSGP

SEQ ID NO: 4 - protein, 14 aa, Homo sapiens
DVSSALDKLKEFGN

SEQ ID NO: 5 - protein, 99 aa, Homo sapiens [sequence omitted]

SEQ ID NO: 6 - protein, 28 aa, Homo sapiens
DHYNCVSSGGQCLYSACPIFTKIQGTCY

SEQ ID NO: 7 - protein, 185 aa, Homo sapiens
MARGAALALLLFGLLGVLVAAPDGGFDLSDALPDNENKKPTAIPKKPSAGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

SEQ ID NO: 8 - protein, 923 aa, Homo sapiens
QYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQ

SEQ ID NO: 9 - protein, 17 aa, Homo sapiens
SSKITHRIHWESASLLR

SEQ ID NO: 10 - protein, 60 aa, Homo sapiens
TTEERGLNVTLSSTGRNGFKSHALQLNNRQIRGLEEELQFSLGSKINVKVGGNSKGTLKV

SEQ ID NO: 11 - protein, 16 aa, Homo sapiens
NGFKSHALQLNNRQIR

SEQ ID NO: 12 - protein, 13 aa, Homo sapiens
NGFKSHALQLNNR

SEQ ID NO: 13 - protein, 11 aa, Homo sapiens
HALQLNNRQIR

SEQ ID NO: 14 - protein, 12 aa, Homo sapiens
SHALQLNNRQIR

SEQ ID NO: 15 - protein, 25 aa, Homo sapiens
DEAGSEADHEGTHSTKRGHAKSRPV

SEQ ID NO: 16 - protein, 25 aa, Homo sapiens
SSSYSKQFTSSTSYNRGDSTFESKS

SEQ ID NO: 17 - protein, 42 aa, Homo sapiens
QGVNDNEEGFFSARGHRPLDKKREEAPSLRPAPPPISGGGYR

SEQ ID NO: 18 - protein, 109 aa, Homo sapiens [sequence omitted]

SEQ ID NO: 19 - protein, 16 aa, Homo sapiens
KEQGHTRRHDWGHEKQ

SEQ ID NO: 20 - protein, 19 aa, Homo sapiens
GHGLGHGHEQQHGLGHGHK

SEQ ID NO: 21 - protein, 9 aa, Homo sapiens
RPPGFSPFR

SEQ ID NO: 22 - protein, 18 aa, Homo sapiens
HGLGHGHEQQHGLGHGHK

SEQ ID NO: 23 - protein, 39 aa, Homo sapiens
AVADTRDQADGSRASVDSGSSEEQGGSSRALVSTLVPLG

SEQ ID NO: 24 - protein, 48 aa, Homo sapiens
DPRLFAEEKAVADTRDQADGSRASVDSGSSEEQGGSSRALVSTLVPLG

SEQ ID NO: 25 - protein, 39 aa, Homo sapiens
DPRLFAEEKAVADTRDQADGSRASVDSGSSEEQGGSSRA

SEQ ID NO: 26 - protein, 18 aa, Homo sapiens
NGAPGIAGAPGFPGARGP

SEQ ID NO: 27 - protein, 27 aa, Homo sapiens
TGLPGPPGERGGPGSRGFPGADGVAGP

SEQ ID NO: 28 - protein, 21 aa, Homo sapiens
SGEPGKQGPSGASGERGPPGP

SEQ ID NO: 29 - protein, 30 aa, Homo sapiens
RGPPGPPGKNGDDGEAGKPGRPGERGPPGP

SEQ ID NO: 30 - protein, 25 aa, Homo sapiens
TSGHPGSPGSPGYQGPPGEPGQAGP

SEQ ID NO: 31 - protein, 33 aa, Homo sapiens
ARGNDGARGSDGQPGPPGPPGTAGFPGSPGAKG

SEQ ID NO: 32 - protein, 33 aa, Homo sapiens
LRGGAGPPGPEGGKGAAGPPGPPGAAGTPGLQG

SEQ ID NO: 33 - protein, 25 aa, Homo sapiens
EPAIIEPGMLIEGPPGPEGPAGLPG

SEQ ID NO: 34 - protein, 115 aa, Homo sapiens
GIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALR

SEQ ID NO: 35 - protein, 14 aa, Homo sapiens
KFLASVSTVLTSKY

SEQ ID NO: 36 - protein, 13 aa, Homo sapiens
TALVELVKHKPKA

SEQ ID NO: 37 - protein, 20 aa, Homo sapiens
DAHKSEVAHRFKDLGEENFK

SEQ ID NO: 38 - protein, 29 aa, Homo sapiens
AAPGQEPPEHMAELQRNEQEQPLGQWHLS

SEQ ID NO: 39 - protein, 30 aa, Homo sapiens
GESHLFRVPSYQALLRGVFHQTVSRKVALG

SEQ ID NO: 40 - protein, 27 aa, Homo sapiens
GSSGGSGAKPSDAASEAARPATSTLNR

SEQ ID NO: 41 - protein, 33 aa, Homo sapiens
GDSTFESKSYKMADEAGSEADHEGTHSTKRGHA

SEQ ID NO: 42 - protein, 27 aa, Homo sapiens
FDESGKGKGIAGRQDILDDSGYVSAYK

SEQ ID NO: 43 - protein, 28 aa, Homo sapiens
SDAAVDTSSEITTKDLKEKKEVVEEAEN

SEQ ID NO: 44 - protein, 17 aa, Homo sapiens
TLPTKETIEQEKRSEIS

SEQ ID NO: 45 - protein, 35 aa, Homo sapiens
SAVPPGADKKAEAGLGQQPEFQFRGGFGRGRGQPP

SEQ ID NO: 46 - protein, 13 aa, Homo sapiens
DGLPGPPGSPGPP